(19) United States
(12) Patent Application Publication
(10) Pub. No.: US 2023/0047389 A1
(43) Pub. Date: Feb. 16, 2023

(54) SAMPLE ANALYSIS DEVICE

(71) Applicant: PHC Holdings Corporation, Tokyo (JP)

(72) Inventors: Kazuya KONDOH, Ehime (JP); Fusatoshi OKAMOTO, Ehime (JP)

(21) Appl. No.: 17/792,951

(22) PCT Filed: Jan. 14, 2021
(86) PCT No.: PCT/JP2021/001096

§ 371 Date: Jul. 14, 2022

(30) Foreign Application Priority Data
Jan. 17, 2020 (JP) 2020-005738
Jan. 17, 2020 (JP) 2020-005739

(51) Int. Cl. G01N 33/543; G01N 35/02; G01N 21/76; B01L 3/00

(52) U.S. Cl. CPC G01N 33/54386; G01N 35/025; G01N 21/76; G01N 33/54326; B01L 3/502761; B01L 2300/0663; B01L 2200/0652

(57) ABSTRACT

A sample analysis substrate mountable and detachable to a sample analysis device and includes: a plate-shaped base substrate; and a chamber, the chamber being a space in which to cause a binding reaction, The sample analysis device includes: a motor to rotate the sample analysis substrate; a first magnet unit to attract the magnetic particles; a first actuator to move the first magnet unit to change relative positions of the first magnet unit and the sample analysis substrate; and a control circuit to control the motor, the drive circuit, and the first actuator. The first magnet unit shaped as a whole shape or a partial shape of a circle or a ring. During a B/F separation for separating reacted substance from unreacted substance, the first actuator moves the first magnet unit to a position where the magnetic particles in the chamber are attracted by the first magnet unit.

### TECHNICAL FIELD

The present application relates to a sample analysis device.

### BACKGROUND ART

Techniques have been known which utilize a sample analysis substrate in order to analyze a specific component within a sample such as urine or blood. For example, Patent Document 1 discloses a technique that utilizes a disk-shaped sample analysis substrate, on which channels, chambers, and the like are formed; in this technique, the sample analysis substrate is allowed to rotate, etc., thereby effecting transfer, distribution, mixing of solutions, analysis of components within sample solution, and so on. The specific component is quantified by detecting light which is generated through immunoreaction, for example.

In immunoassay techniques and genetic detection techniques, magnetic particles (which may also be referred to as “magnetic beads”, “magnetism particles”, “magnetism beads”, etc.) are used, for example. Patent Document 2 refers to a sandwich immunoassay using magnetic particles. In a sandwich immunoassay, through an antigen-antibody reaction, an antigen that is contained in a sample for measurement, a primary antibody immobilized to the surface of magnetic particles, and a secondary antibody having a label substance bound thereto are bound to produce a composite. The antigen-antibody reaction requires a step of B/F separation (Bound/Free Separation). The B/F separation step includes steps of capturing the magnetic particles by using a magnet(s), and removing liquid (specimen solution, reagent solution, wash solution, etc.) and washing the magnetic particles to separate reacted substance from unreacted substance and remove the unreacted substance. A B/F separation using a magnet(s) and magnetic particles is necessary for not only those immunoassay techniques which are based on a non-competitive assay but also those which are based on a competitive assay, as well as genetic detection techniques based on hybridization.

In order to effect B/F separation, the sample analysis substrate includes a magnet in Patent Document 2. The magnet may be non-removable or removable with respect to the sample analysis substrate. A balancer is also attached to the sample analysis substrate in order to suppress shifts in the center of gravity associated with rotation.

### CITATION LIST

### Patent Literature

[Patent Document 1] Japanese National Phase PCT Laid-Open Publication No. H7-500910

[Patent Document 2] Japanese Laid-Open Patent Publication No. 2018-163102

### SUMMARY OF INVENTION

### Technical Problem

Generally, sample analysis substrates are disposable. If a magnet and a balancer are non-removably attached to the sample analysis substrate, the magnet and balancer are thrown away together with the sample analysis substrate. Therefore, the costs associated with the magnet and balancer are incurred each time, thus increasing the cost of the sample analysis substrate.

If the magnet and balancer are removable from the sample analysis substrate, the magnet and balancer are not thrown away each time. However, costs are incurred for the operations and management, such as attachment and detachment, washing, storage, etc., of the magnet and balancer.

Therefore, an analytical environment is needed that allows samples to be analyzed at low cost. A non-limiting and illustrative embodiment of the present application provides a sample analysis device that can suppress costs.

### Solution to Problem

A sample analysis device according to the present disclosure is a sample analysis device that rotates and stops a sample analysis substrate retaining a liquid sample to cause a binding reaction between an analyte in the liquid sample and a ligand immobilized to surfaces of magnetic particles, the sample analysis device including: a turntable to support the sample analysis substrate mounted thereon; a motor to rotate the turntable; a drive circuit to control rotation and stopping of the motor; a first magnet unit to generate a force for attracting the magnetic particles; a first actuator to move the first magnet unit to change relative positions of the first magnet unit and the sample analysis substrate; and a control circuit to control operation of the motor, the drive circuit, and the first actuator. The sample analysis substrate being capable of being mounted to or detached from the sample analysis device and includes: a plate-shaped base substrate having a predetermined thickness; and a chamber within the base substrate, the chamber being a space in which to cause the binding reaction. The first magnet unit has a first shape that is a whole shape or a partial shape of a circle or a ring. During a B/F separation (Bound/Free Separation) for separating reacted substance from unreacted substance within the chamber, the first actuator moves the first magnet unit to a position where the magnetic particles in the chamber are attracted by the first magnet unit.

### Advantageous Effects of Invention

According to the present disclosure, there is provided a sample analysis device that suppresses costs. Moreover, a sample analysis device that is capable of enhancing the measurement accuracy for a specific component in a sample is provided.

### BRIEF DESCRIPTION OF DRAWINGS

FIG. 1 is an exemplary schematic diagram describing a sandwich immunoassay that utilizes magnetic particles.

FIG. 2A is a plan view showing an example structure of a sample analysis substrate.

FIG. 2B is an exploded perspective view of the sample analysis substrate.

FIG. 3 is a block diagram showing an example hardware configuration of a sample analysis device 1.

FIG. 4A is a plan view of a sample analysis substrate 100.

FIG. 4B is an exploded perspective view of the sample analysis substrate 100.

FIG. 5 is a top view showing the positions of a plurality of chambers provided on the sample analysis substrate 100.

FIG. 6 is a top view showing the positions of a wash solution 130, a substrate solution 132, a primary antibody 134, and a secondary antibody 136, which are previously retained in the sample analysis substrate 100.

FIG. 7 is a diagram showing an application chamber 110 in which blood 190, being a specimen, has been applied dropwise.

FIG. 8 is an exploded perspective view of a first magnet unit 16.

FIG. 9 is a plan view of the first magnet unit 16.

FIG. 10A is a diagram showing an example shape of a magnet according to the present disclosure.

FIG. 10B is a diagram showing an example shape of a magnet according to the present disclosure.

FIG. 10C is a diagram showing an example shape of a magnet according to the present disclosure.

FIG. 10D is a diagram showing an example shape of a magnet according to the present disclosure.

FIG. 10E is a diagram showing an example shape of magnets according to the present disclosure.

FIG. 10F is a diagram showing an example shape of magnets according to the present disclosure.

FIG. 10G is a diagram showing an example shape of magnets according to the present disclosure.

FIG. 10H is a diagram showing an example shape of magnets according to the present disclosure.

FIG. 11 is a plan view showing a semicircular-ring shaped first magnet unit 16 having moved to above a circular sample analysis substrate 100, and the construction of a moving mechanism for the first magnet unit 16.

FIG. 12 is a side view showing a semicircular-ring shaped first magnet unit 16 having moved to above the circular sample analysis substrate 100, and the construction of the moving mechanism for the first magnet unit 16.

FIG. 13 is a diagram showing a relationship between the position of the first magnet unit 16 and the position of a measurement chamber 116 after the sample analysis substrate 100 has been rotated by about 180°.

FIG. 14 shows an A-A cross section in FIG. 13.

FIG. 15 is a plan view showing the first magnet unit 16 having been moved to a position retracted from above the sample analysis substrate 100, and the construction of the moving mechanism for the first magnet unit 16.

FIG. 16 is a side view showing the first magnet unit 16 having been moved to a position retracted from above the sample analysis substrate 100, and the construction of the moving mechanism for the first magnet unit 16.

FIG. 17 is an enlarged view of a B-B cross section in FIG. 15.

FIG. 18 is a flowchart showing a procedure of processing by a control circuit 22 during a B/F separation process.

FIG. 19 is a plan view showing the construction of semicircular-ring shaped first magnet units 16 and 56 and moving mechanisms for moving the first magnet units 16 and 56.

FIG. 20 is a side view showing the construction of semicircular-ring shaped first magnet units 16 and 56 and moving mechanisms for moving the first magnet units 16 and 56.

FIG. 21 shows a second magnet unit 56 having moved away from the sample analysis substrate 100.

FIG. 22 is a side view for describing a modification concerning the moving directions of the first magnet unit 16.

FIG. 23 is a block diagram showing an example hardware configuration of a sample analysis device 1.

FIG. 24 is a diagram illustrating an example relative positioning between the first magnet unit 16, the second magnet unit 56, and the sample analysis substrate 100.

FIG. 25 is a diagram illustrating an example relative positioning between the first magnet unit 16, the second magnet unit 56, and the sample analysis substrate 100.

FIG. 26 is a diagram showing a relationship between the position of the first magnet unit 16 and the position of the measurement chamber 116 after the sample analysis substrate 100 has been rotated by about 180° from the state shown in FIG. 24.

FIG. 27 is an enlarged view of an A-A cross section in FIG. 26.

FIG. 28 is a plan view showing the first magnet unit 16 having been moved to a position retracted from above the sample analysis substrate 100, and the construction of the moving mechanism for the first magnet unit 16.

FIG. 29 is a side view showing the first magnet unit 16 having been moved to a position retracted from above the sample analysis substrate 100, and the construction of the moving mechanism for the first magnet unit 16.

FIG. 30 is a plan view showing the second magnet unit 56 having moved to a position overlapping the sample analysis substrate 100 and the construction of the moving mechanism for the second magnet unit 56.

FIG. 31 is a side view showing the second magnet unit 56 having moved to a position overlapping the sample analysis substrate 100 and the construction of the moving mechanism for the second magnet unit 56.

FIG. 32 is an enlarged view of a C-C cross section in FIG. 30.

FIG. 33 is a flowchart showing a procedure of processing by the control circuit 22 of carrying out an agitation process utilizing magnetic particles.

FIG. 34 is a flowchart showing a procedure of processing by the control circuit 22 carrying out a luminescence measurement process.

FIG. 35 is a diagram showing an example relative positioning between a ring-shaped first magnet unit 16, a semicircular-shaped second magnet unit 56, and the sample analysis substrate 100.

FIG. 36A is a side view of a sample analysis device 6 according to a modification.

FIG. 36B is a diagram showing S-poles of magnets 40 and 80 facing each other.

FIG. 37 is a side view of a sample analysis device 6 according to a further modification.

### DESCRIPTION OF EMBODIMENTS

Hereinafter, with reference to the attached drawings, embodiments of sample analysis devices according to the present invention will be described. Note however that unnecessarily detailed descriptions may be omitted in the present specification. For example, detailed descriptions on what is well known in the art or redundant descriptions on what is substantially the same constitution may be omitted. This is to avoid lengthy description, and facilitate the understanding of those skilled in the art. The accompanying drawings and the following description, which are provided by the inventors so that those skilled in the art can sufficiently understand the present disclosure, are not intended to limit the scope of claims. In the present specification, identical or similar constituent elements are denoted by identical reference numerals.

Assay techniques for components within a sample such as urine or blood may utilize a binding reaction between an analyte being the subject for analysis and a ligand which specifically binds to the analyte. Examples of such assay techniques include immunoassay techniques and genetic diagnosis techniques. A sample such as urine or blood may be referred to as a specimen in the fields of medicine and pharmacy.

Examples of immunoassay techniques are competitive assays and non-competitive assays (sandwich immunoassay). Examples of genetic diagnosis techniques are genetic detection techniques based on hybridization. In these immunoassay techniques and genetic detection techniques, magnetic particles (which may also be referred to as “magnetic beads”, “magnetism particles”, “magnetism beads”, etc.) are used, for example. As an example of such assay techniques, a sandwich immunoassay utilizing magnetic particles will be specifically described.

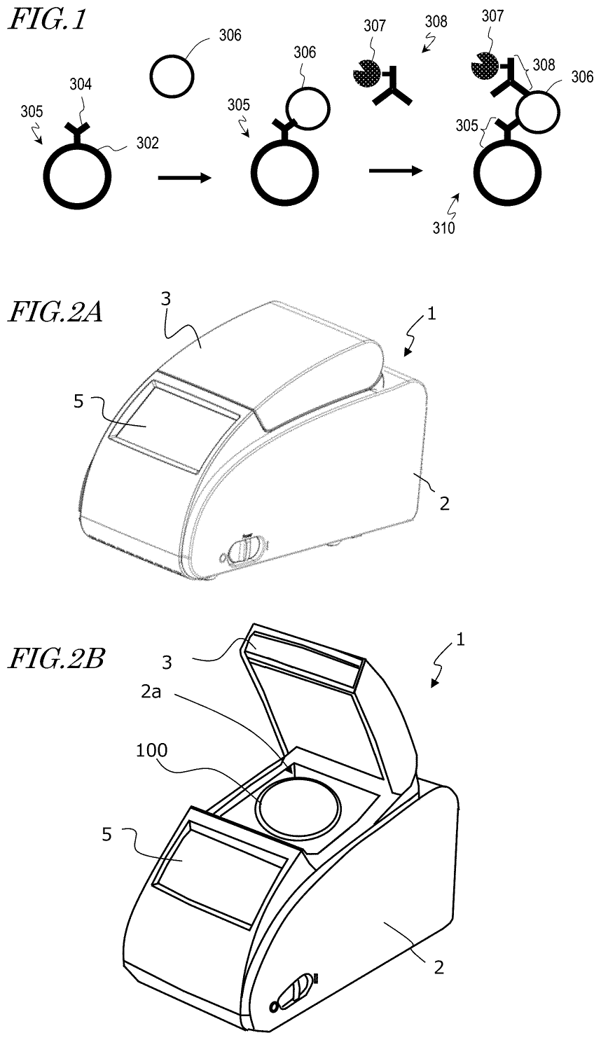

As shown in FIG. 1, first, a primary antibody 304 immobilized to the surface of magnetic particles 302 (hereinafter referred to as a “magnetic-particle-immobilized antibody 305”) and an antigen 306 contained in a sample for measurement are allowed to bind through an antigen-antibody reaction. Next, a secondary antibody having a label substance 307 bound thereto (hereinafter referred to as a “labeled antibody 308”) and the antigen 306 are allowed to bind through an antigen-antibody reaction. As a result, a composite 310 is obtained in which the magnetic-particle-immobilized antibody 305 and the labeled antibody 308 are bound to the antigen 306.

A signal which is based on the label substance 307 of the labeled antibody 308 that has bound to the composite 310 is detected, and an antigen concentration is measured in accordance with the amount of detected signal. Examples of the label substance 307 include enzymes (e.g., peroxidase, alkaline phosphatase, and luciferase), chemiluminescent substances, electrochemiluminescent substances, and fluorescent substances. In accordance with each such label substance 307, dye, luminescence, fluorescence, or other signals are detected. Although the light to be detected is not emitted from the sample itself, component analysis of the sample consists in measuring the concentration of the antigen 306 or the like within the sample, and it is the composite 310 with the antigen 306 having bound thereto that undergoes luminescence; therefore, for ease of understanding, the sample will be said to be undergoing luminescence in the present specification.

When using the aforementioned measurement method to transfer a sample among a plurality of chambers provided on a sample analysis substrate and to perform a component analysis for the sample by detecting luminescence of the sample, there may be cases where luminescence of the sample is detected while keeping the sample analysis substrate rotated, this being in order to transfer or retain the sample through utilization of the order of transfers of the sample or a centrifugal force caused by rotation of the sample analysis substrate.

The aforementioned luminescence measurement is aimed at the reaction solution remaining after the unreacted substance which has not undergone an antigen-antibody reaction is removed. Therefore, this requires a step of removing or separating the unreacted substance, i.e., a B/F separation (Bound/Free Separation) step. As used herein, the “reacted substance” is the composite. The “unreacted substance” is, for example: unreacted substance in the specimen; any substance that has non-specifically adsorbed to magnetic particles or the like; and any label substance that was not involved in the production of the composite.

According to an embodiment of the present disclosure, a magnet for removing the unreacted substance is provided not on the sample analysis substrate but on the sample analysis device. In other words, sample analysis devices according to the present disclosure can be summarized as follows.

### [Item 1]

A sample analysis device that rotates and stops a sample analysis substrate retaining a liquid sample to cause a binding reaction between an analyte in the liquid sample and a ligand immobilized to surfaces of magnetic particles,

the sample analysis substrate being capable of being mounted to or detached from the sample analysis device and including: a plate-shaped base substrate having a predetermined thickness; and a chamber within the base substrate, the chamber being a space in which to cause the binding reaction,

wherein the sample analysis device comprises:

a turntable to support the sample analysis substrate mounted thereon;

a motor to rotate the turntable;

a drive circuit to control rotation and stopping of the motor;

a first magnet unit to generate a force for attracting the magnetic particles;

a first actuator to move the first magnet unit to change relative positions of the first magnet unit and the sample analysis substrate; and

a control circuit to control operation of the motor, the drive circuit, and the first actuator,

wherein the first magnet unit is shaped as a whole shape or a partial shape of a circle or a ring; and,

during a B/F separation (Bound/Free Separation) for separating reacted substance from unreacted substance within the chamber, the first actuator moves the first magnet unit to a position where the magnetic particles in the chamber are attracted by the first magnet unit.

### [Item 2]

The sample analysis device of Item 1, wherein the first magnet unit comprises a single magnet having the shape or a plurality of magnets arranged along the shape.

### [Item 3]

The sample analysis device of Item 1 or 2, wherein,

the sample analysis substrate is circular; and

the first magnet unit is shaped as a whole or a part of the circle or the ring such that a sum of central angles thereof is not less than 90 degrees and not more than 360 degrees.

### [Item 4]

The sample analysis device of any of Items 1 to 3, wherein,

the sample analysis substrate is circular, and the first magnet unit is shaped as a part of the circle or the ring; and

a length along a circumferential direction of the first magnet unit is longer than a length along a circumferential direction of the chamber.

s[Item 5]

The sample analysis device of any of Items 1 to 4, wherein,

the sample analysis substrate is circular; and

a radius size of the circle or the ring is determined in accordance with a distance from a center of rotation of the sample analysis substrate to the chamber.

### [Item 6]

The sample analysis device of any of Items 1 to 5, wherein,
the first magnet unit is shaped as a whole or a part of the ring; and
the first actuator moves the first magnet unit during the B/F (Bound/Free) separation so that a central position regarding a radial direction of the ring matches a position in the chamber that is the farthest from the center of rotation of the sample analysis substrate.

### [Item 7]

The sample analysis device of any of Items 1 to 6, wherein the first actuator moves the first magnet unit along a direction that is parallel to a rotation axis of the sample analysis substrate.

### [Item 8]

The sample analysis device of any of Items 1 to 6, wherein the first actuator moves the first magnet unit along a direction that is perpendicular to the rotation axis of the sample analysis substrate.

### [Item 9]

The sample analysis device of Item 8, wherein the first actuator moves the first magnet unit to a position at which the first magnet unit and the sample analysis substrate do not overlap as viewed from a direction that is parallel to the rotation axis of the sample analysis substrate.

### [Item 10]

The sample analysis device of any of Items 1 to 9, wherein the first magnet unit is located on an opposite side of the sample analysis substrate from the turntable.

### [Item 11]

The sample analysis device of any of Items 1 to 9, wherein the first magnet unit is located on a same side of the sample analysis substrate as the turntable.

### [Item 12]

The sample analysis device of any of Items 1 to 11, wherein
the first magnet unit is a first magnet unit, and the first actuator is a first actuator,
the sample analysis device further comprising:
a second magnet unit distinct from the first magnet unit; and
a second actuator to move the second magnet unit along a direction that is perpendicular to the rotation axis of the sample analysis substrate to change relative positions of the second magnet unit and the sample analysis substrate.

### [Item 13]

The sample analysis device of Item 12, wherein the second magnet unit comprises a single magnet having a whole shape or a partial shape of a circle or a ring, or a plurality of magnets arranged along the shape.

### [Item 14]

The sample analysis device of any of Items 1 to 13, wherein the first actuator is a stepping motor or a linear motor.

### [Item 15]

The sample analysis device of Item 12 or 13, wherein the first actuator and the second actuator are a stepping motor(s) or a linear motor(s).

### [Item 16]

The sample analysis device of any of Items 12 to 15, wherein the first magnet unit is located on an opposite side of the sample analysis substrate from the turntable; and
the second magnet unit is located on a same side of the sample analysis substrate as the turntable.

### [Item 17]

A sample analysis device that rotates and stops a sample analysis substrate retaining a liquid sample to cause a binding reaction between an analyte in the liquid sample and a ligand immobilized to surfaces of magnetic particles,

the sample analysis substrate being capable of being mounted to or detached from the sample analysis device and including: a plate-shaped base substrate having a predetermined thickness; and a chamber within the base substrate, the chamber being a space in which to cause the binding reaction,

wherein the sample analysis device comprises:

a turntable to support the sample analysis substrate mounted thereon;

a motor to rotate the turntable;

a drive circuit to control rotation and stopping of the motor;

a first magnet unit to generate an attractive force for attracting the magnetic particles, the first magnet unit being disposed at a first face that is perpendicular to the rotation axis of the sample analysis substrate;

a second magnet unit to generate an attractive force for attracting the magnetic particles, the second magnet unit being disposed at a second face that is perpendicular to the rotation axis of the sample analysis substrate, the second face being opposite to the first face;

a first actuator to move the first magnet unit to change relative positions of the first magnet unit and the sample analysis substrate; and

a second actuator to move the second magnet unit to change relative positions of the second magnet unit and the sample analysis substrate; and

a control circuit to control operation of the motor, the drive circuit, the first actuator, and the second actuator, wherein,

during agitation of the liquid sample in the chamber, the first actuator and the second actuator alternately move the first magnet unit and the second magnet unit to a position where the magnetic particles in the chamber are attracted by the first magnet unit and the second magnet unit.

### [Item 18]

The sample analysis device of Item 17, wherein,

the first face is a face that is opposite to the turntable with respect to the sample analysis substrate;

the first magnet unit has a first shape that is a whole or a part of a circle or a ring; and

the second magnet unit has a second shape that is a partial shape of a circle or a whole shape or a partial shape of a ring.

### [Item 19]

The sample analysis device of Item 18, wherein,

the first magnet unit comprises a single magnet having the first shape or a plurality of magnets arranged along the first shape; and

the second magnet unit comprises a single magnet having the second shape or a plurality of magnets arranged along the second shape.

### [Item 20]

The sample analysis device of Item 18 or 19, wherein,

in a case where movement of the magnetic particles requires T seconds when the sample analysis substrate rotates at a predetermined number of revolutions and the magnetic particles are attracted at the number of revolutions;

in a period of 2T seconds, the first actuator causes the first magnet unit to approach the sample analysis substrate and move away from the sample analysis substrate, and,

in a period of 2T seconds, the second actuator causes the second magnet unit to move away from the sample analysis substrate and approach the sample analysis substrate.

### [Item 21]

The sample analysis device of any of Items 18 to 20, wherein,

the first shape and the second shape are a whole or a part of a ring;

the first actuator and the second actuator cause the first magnet unit and the second magnet unit, respectively, to approach the sample analysis substrate so that a central position regarding a radial direction of the ring matches a position in the chamber that is the farthest from the center of rotation of the sample analysis substrate.

### [Item 22]

The sample analysis device of any of Items 17 to 21, wherein the first actuator and the second actuator cause the first magnet unit and the second magnet unit, respectively, to move along a direction that is parallel to the rotation axis of the sample analysis substrate.

### [Item 23]

The sample analysis device of any of Items 17 to 21, wherein,

the second shape is a partial shape of the ring; and

the first actuator and the second actuator cause the first magnet unit and the second magnet unit, respectively, to move along a direction that is perpendicular to the rotation axis of the sample analysis substrate.

### [Item 24]

The sample analysis device of Item 23, wherein,

the first actuator causes the first magnet unit to move away to a position at which the first magnet unit and the sample analysis substrate do not overlap as viewed from a direction that is parallel to the rotation axis of the sample analysis substrate; and

the second actuator causes the second magnet unit to move away to a position at which the second magnet unit and the sample analysis substrate do not overlap as viewed from the direction that is parallel to the rotation axis of the sample analysis substrate.

### [Item 25]

The sample analysis device of any of Items 17 to 24, wherein,

the first magnet unit and the second magnet unit face each other with the sample analysis substrate interposed therebetween; and

the first magnet unit and the second magnet unit have mutually opposite polarities on the sample analysis substrate side.

### [Item 26]

The sample analysis device of any of Items 17 to 25, further comprising a photosensor disposed by the second face, wherein,

during a luminescence reaction to be effected by allowing a predetermined luminescent substrate to act on a composite of the analyte and the ligand being bound together after completion of the binding reaction;

the second actuator moves the second magnet unit to a position where the magnetic particles in the chamber are attracted by the second magnet unit; and

the photosensor detects light generated from the luminescence reaction.

### [Item 27]

The sample analysis device of Item 26, wherein the photosensor is a photomultiplier tube.

### [Item 28]

The sample analysis device of any of Items 17 to 27, wherein the first actuator and the second actuator are a stepping motor(s) or a linear motor(s).

In accordance with the aforementioned illustrative implementation, the magnet(s) utilized for B/F separation is provided not on a disposable sample analysis substrate, but on a sample analysis device. Since the magnet(s) is not thrown away with the sample analysis substrate, and there is no need to provide a balancer on the sample analysis substrate, costs for the sample analysis substrate can be reduced. Therefore, a sample analysis device that suppresses costs is provided.

Moreover, in accordance with the aforementioned illustrative implementation, the first magnet unit and the second magnet unit are disposed on, respectively, a first face of the sample analysis substrate and a second face that is opposite to the first face. During agitation of the liquid sample in the chamber of the sample analysis substrate, the sample analysis device alternately moves the first magnet unit or the second magnet unit to a position where the magnetic particles in the chamber are attracted by the magnet unit. When the first magnet unit moves so as to come closer to the sample analysis substrate, the magnetic particles are attracted toward the first face; when the second magnet unit moves so as to come closer to the sample analysis substrate, the magnetic particles are attracted toward the second face. Because the liquid sample is agitated with the movement of magnetic particles in the chamber, an antigen-antibody reaction can be caused while suppressing unevenness of reaction. This allows to enhance the measurement accuracy for a specific component in a sample. When an antigen-antibody reaction is caused between a sample and a reagent, for example, the reaction can be promoted by agitating the inside of the chamber by performing the aforementioned operation. Moreover, washing of a solution can be achieved by performing the aforementioned operation in a B/F separation step, for example.

Hereinafter, sample analysis devices according to illustrative embodiments of the present disclosure will be described.

### First Embodiment

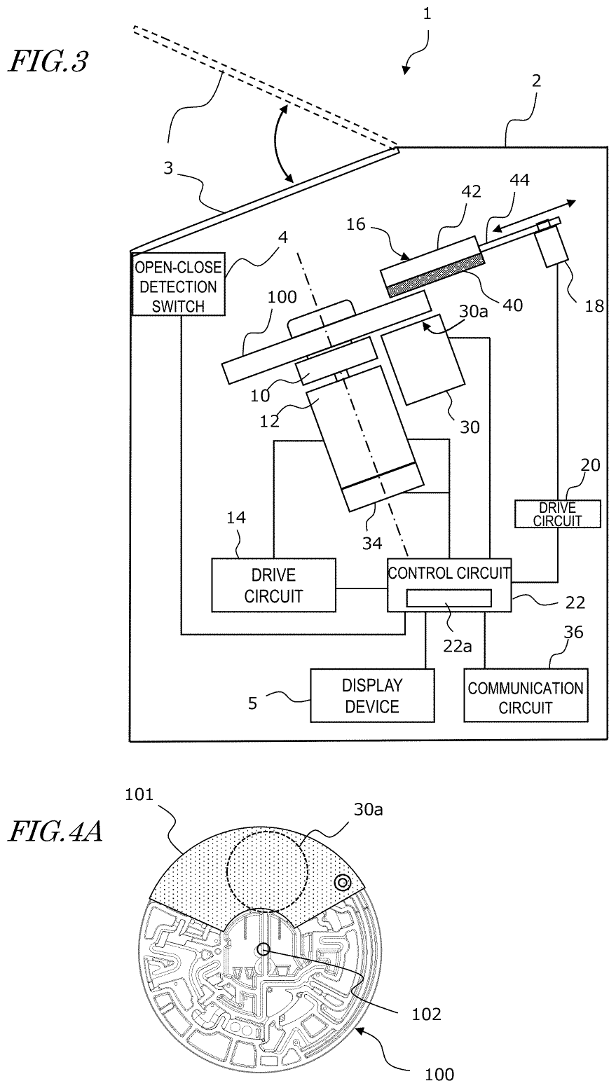

FIG. 2A and FIG. 2B are perspective views showing the appearance of a sample analysis device 1 according to an illustrative first embodiment of the present disclosure. FIG. 3 is a block diagram showing an example hardware configuration of the sample analysis device 1.

The sample analysis device 1 rotates and stops a sample analysis substrate 100 retaining a liquid sample to cause a binding reaction between an analyte in the liquid sample and a ligand immobilized to the surfaces of magnetic particles.

The sample analysis device 1 has a housing 2 that includes a door 3 which is capable of opening and closing. The housing 2 has an accommodation 2a in which the sample analysis substrate 100 is accommodated so as to be capable of rotation, such that a motor 12 having a turntable 10 is disposed in the accommodation 2a. While the door 3 is open, the sample analysis substrate 100 can be attached to or detached from the turntable 10 within the accommodation 2a. As the door 3 is closed, the door 3 shields the accommodation 2a from light so that no light may enter the accommodation 2a from the exterior. On the housing 2, a display device 5 for displaying analysis results is provided.

Hereinafter, the construction of the sample analysis substrate 100 will be described first. In the present embodiment, blood is what is to be analyzed by using the sample analysis substrate 100. The sample analysis substrate 100 also includes chambers and reagents which are suitable for blood analysis. Note that the sample analysis substrate 100 according to the present embodiment lacks magnets and balancers. The magnet(s) is provided on the sample analysis device 1 side.

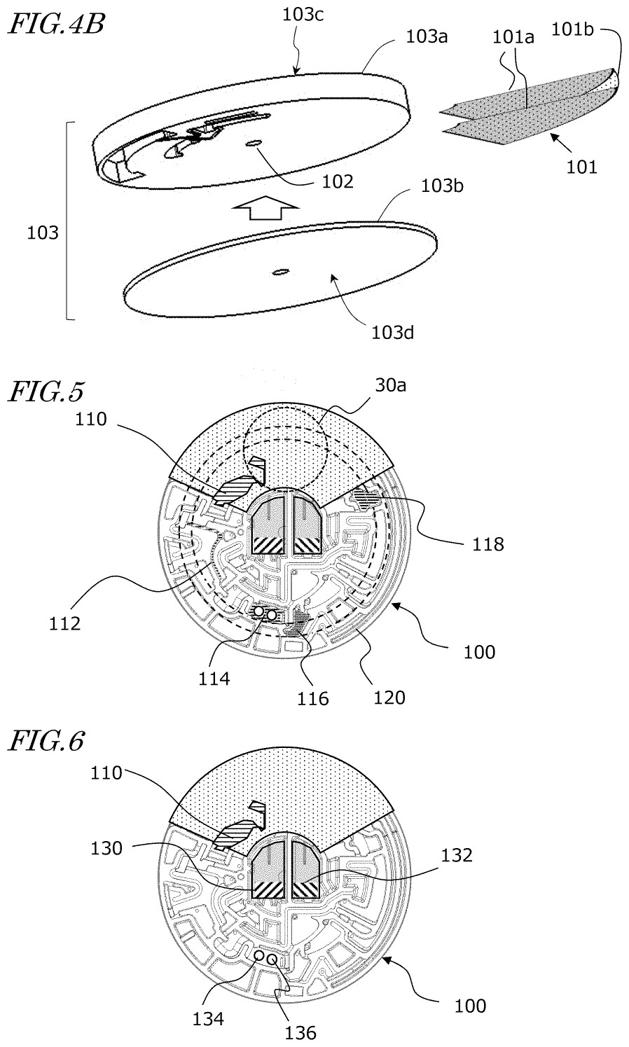

FIG. 4A and FIG. 4B are a plan view and an exploded perspective view, respectively, of the sample analysis substrate 100. The sample analysis substrate 100 includes: a light-shield cap 101; and a plate-shaped substrate 103 having a rotation axis 102 and a predetermined thickness along a direction that is parallel to the rotation axis 102. Although the substrate 103 of the sample analysis substrate 100 has a circular shape in the present embodiment, it may alternatively be shaped as a polygon, an ellipse, a sector, or the like. The substrate 103 has two principal faces 103c and 103d. In the present embodiment, the principal face 103c and the principal face 103d are parallel to each other, and the thickness of the substrate 103 as defined by an interspace between the principal face 103c and the principal face 103d is constant irrespective of position within the substrate 103. However, the principal faces 103c and 103d do not need to be parallel. For example, the two principal faces may be partly non-parallel or parallel, or be entirely non-parallel. Moreover, at least one of the principal faces 103c and 103d of the substrate 103 may have a structure with recesses or protrusions.

The light-shield cap 101, which includes a pair of shading portions 101a and a connecting portion 101b, is attached to the substrate 103 so that the shading portions 101a partially cover the principal faces 103c and 103d of the substrate 103. In the present embodiment, each shading portion 101a has a substantial sector shape. The shading portions 101a are made of a material that does not transmit luminescence occurring from the composite 310. Preferably, each shading portion 101a is provided at a position on the principal faces 103c and 103d of the substrate 103 that is opposed to the light-receiving surface 30a of a photodetector 30. The photodetector 30 is used to detect luminescence from a sample at the measurement chamber 116, whose light-receiving surface 30a is a region to receive light. Moreover, a central angle α of the region of the principal face 103c or the principal face 103d where the shading portion 101a is located is preferably larger than a central angle β of the region where the measurement chamber 116 is located.

The substrate 103 of the sample analysis substrate 100 is composed of a base substrate 103a and a cover substrate 103b.

The sample analysis substrate 100 includes a plurality of chambers located in the substrate 100 and channels connecting between the chambers. The plurality of chambers may be a reaction chamber, a measurement chamber, a substrate retention chamber, and a recovery chamber, for example.

The respective spaces of the plurality of chambers are formed within the base substrate 103a, and as the cover substrate 103b covers over the base substrate 103a, a top and a bottom of each space are created. In other words, the respective spaces of the plurality of chambers are defined by at least one inner face of the sample analysis substrate 100. The channels are also formed in the base substrate 103a, and as the cover substrate 103b covers over the base substrate 103a, a top and a bottom of each space of the respective channel are created. Thus, the chambers and the channels are enclosed within the substrate 103.

FIG. 5 is a top view showing the positions of a plurality of chambers provided on the sample analysis substrate 100. The sample analysis substrate 100 may include an application chamber 110, a plasma quantification chamber 112, a reaction chamber 114, a measurement chamber 116, a substrate retention chamber 118, and a recovery chamber 120, for example. The position of the light-receiving surface 30a of the photodetector 30 is shown also in FIG. 5.

FIG. 6 is a top view showing the positions of a wash solution 130, a substrate solution 132, a primary antibody 134, and a seconpppdary antibody 136, which are previously retained in the sample analysis substrate 100. The primary antibody 134 is the magnetic-particle-immobilized antibody 305. The secondary antibody 136 is the labeled antibody 308. The magnetic-particle-immobilized antibody 305 and the labeled antibody 308 are carried in the reaction chamber 114 in a dry state. These may also be referred to as “dried reagents”.

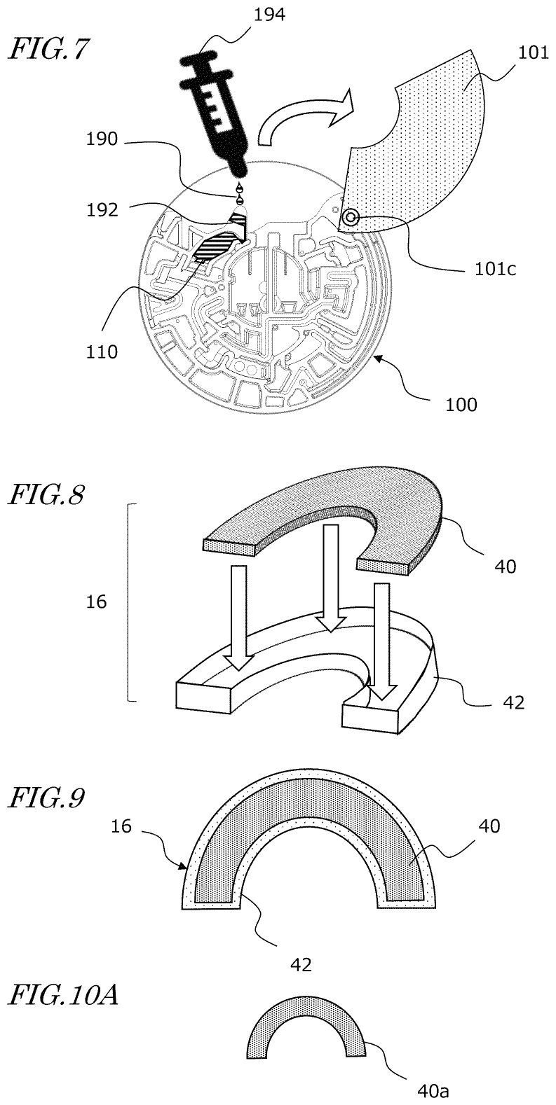

FIG. 7 shows an application chamber 110 in which blood 190, being a specimen, has been applied dropwise. During application, the user rotates the light-shield cap 101 clockwise around a pivot 101c to expose an application point 192. The user may use a syringe 194 to apply blood dropwise from the application point, for example.

The blood 190 is subjected to centrifugal separation through rapid rotations of the sample analysis substrate 100 as caused by the sample analysis device 1. The blood plasma having experienced the centrifugal separation is transferred from the plasma quantification chamber 112 shown in FIG. 5 through a channel, to reach the reaction chamber 114 by means of rotation, swing, and stopping of the rotation of the sample analysis substrate 100 as caused by the sample analysis device 1. The blood plasma is a sample solution containing the antigen 306. In the reaction chamber 114, dried reagents are dissolved by the sample solution, whereby an antigen-antibody reaction (immunoreaction) occurs. This produces the composite 310.

As has been described in the BACKGROUND ART section, when an antigen-antibody reaction occurs, a B/F separation step for separating reacted substance from unreacted substance is required. As used herein, the “reacted substance” is a composite, whereas the “unreacted substance” is, for example, unreacted substance in the specimen, and any label substance that was not involved in the production of the composite.

According to an embodiment of the present disclosure, a magnet is provided on the sample analysis device 1, while the sample analysis substrate 100 requires no magnet or balancer. The sample analysis device 1 controls the magnet to come closer to the sample analysis substrate 100, thereby capturing the magnetic particles and removing unreacted substance.

With reference again to FIG. 3, the hardware configuration of the sample analysis device 1 will be described.

The sample analysis device 1 includes an open-close detection switch 4, the display device 5, the motor 12, drive circuits 14 and 20, a first magnet unit 16, a first actuator 18, a control circuit 22, the photodetector 30, an encoder 34, and a communication circuit 36.

The open-close detection switch 4 is a momentary switch that detects opening and closing of the door 3, for example, but any other switch may be adopted.

The motor 12, which has the turntable 10 supporting the sample analysis substrate 100 mounted thereon, and rotates the sample analysis substrate 100 around the rotation axis 102. The rotation axis 102 may be inclined from the direction of gravity at an angle of not less than 0° and not more than 90° with respect to the direction of gravity. The motor 12 may rotate the sample analysis substrate 100 in a range from 100 rpm to 8000 rpm, for example. The rotational speed may be determined in accordance with the shape of each chamber and channel, the physical properties of liquids, the timing of transfers and treatments of liquids, and the like. The motor 12 may be a DC motor, a brushless motor, an ultrasonic motor, or the like, for example.

The drive circuit 14 controls rotation and stopping of the motor 12. Specifically, based on a command from the control circuit 22, the drive circuit 14 rotates the sample analysis substrate 100 clockwise or counterclockwise, swings it, and controls stopping of the rotation and the swing.

The first magnet unit 16 includes one or more magnets, and with the one or more magnets, generates a force (magnetic force) to attract the magnetic particles. The first magnet unit 16 has a “whole or partial” shape of “a circle or a ring”. A “whole or partial” shape of “a circle or a ring” is achieved by the shape of a single magnet or an arrangement of a plurality of magnets. The specific construction of the first magnet unit 16 will be described later. To the first magnet unit 16, a first rack 44 having teeth thereon is attached.

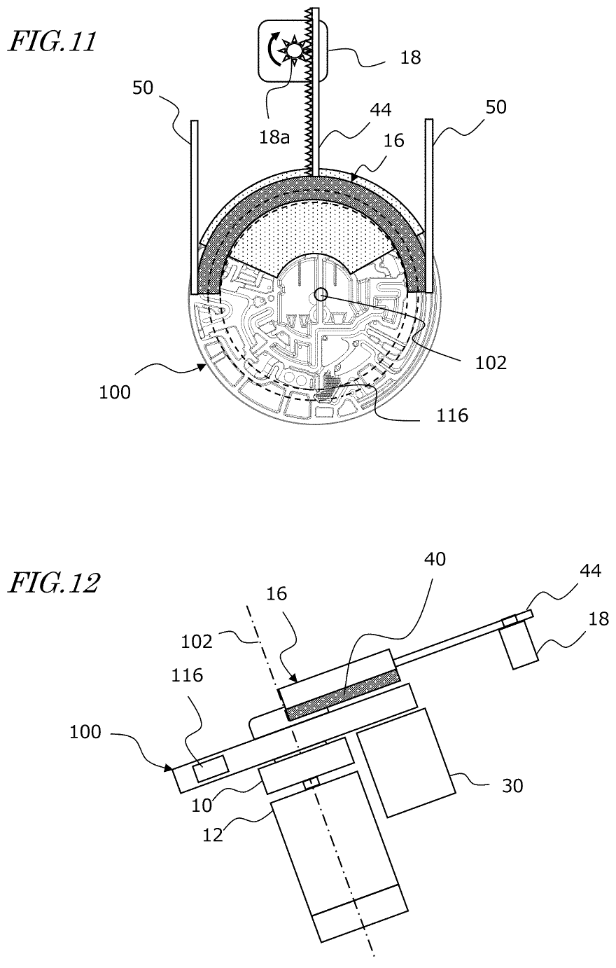

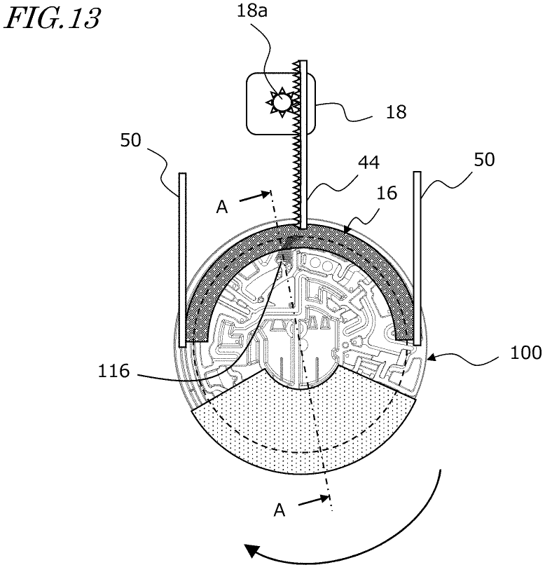

The first actuator 18 moves the first magnet unit 16 by moving the first rack 44 along the longitudinal direction, thereby changing the relative positions of the first magnet unit 16 and the sample analysis substrate 100. The operation of the first actuator 18 is controlled by the drive circuit 20. An example of the first actuator 18 is an electric motor that undergoes rotational motion. The first actuator 18 may be a stepping motor or a linear motor, for example. Details of the construction and operation regarding the first actuator 18 will be described later with reference to FIG. 11, FIG. 12, and so on.

The control circuit 22 controls the operation of the motor 12, the first actuator 18, and the drive circuits 14 and 20.

The photodetector 30 detects luminescence occurring from the label substance 307 of the labeled antibody 308 bound to the composite 310 (FIG. 1) being retained in the measurement chamber 116 (FIG. 5) of the sample analysis substrate 100. Herein, luminescence refers to any release of photons, irrespective of the principle of luminescence, e.g., fluorescence or phosphorescence. That is, the photodetector 30 measures a number of photons in the luminescence occurring from the label substance 307 and striking the light-receiving surface 30a.

With the sample analysis substrate 100 being attached to the turntable 10, the light-receiving surface 30a of the photodetector 30 is disposed below a concentric circle on which the measurement chamber 116 is located, i.e., on the same side of the sample analysis substrate 100 as the turntable 10.

The photodetector 30 may be a photomultiplier tube that includes a lens shutter and a photon counter (neither of which is shown), for example. The lens shutter is provided between the light-receiving surface 30a of the photodetector 30 and the sample analysis substrate 100, and controls opening and closing of the light-receiving surface 30a. While the shutter is open, luminescence occurring from the composite 310 being retained in the measurement chamber 116 of the rotating sample analysis substrate 100 is incident on the light-receiving surface 30a. While the shutter is closed, luminescence is blocked. The shutter may have a mechanical structure, or be a liquid crystal shutter or the like. At the light-receiving surface 30a, the photomultiplier tube receives photons of luminescence occurring from the label substance 307 and counts pulses of which there are as many as the photons, and outputs the count.

By associating the count of photons with the rotation angle of the sample analysis substrate 100, the control circuit 22 generates a photon count distribution signal.

The encoder 34 is a so-called rotary encoder that is attached to the shaft of the motor 12 and detects the rotation angle of the motor 12. When the sample analysis substrate 100 is attached to the turntable 10, the sample analysis substrate 100 rotates around the rotation axis 102, and therefore the output of the encoder 34 can be utilized as a rotation angle signal, in which the rotation angle of the sample analysis substrate 100 is detected. The rotation angle signal may be a pulse signal containing pulses that are output for every predetermined angle, for example. In the case where the motor 12 is a brushless motor, it may be possible to adopt, instead of the encoder 34: a Hall generator provided in the brushless motor; and a detection circuit that receives an output signal from the Hall generator and outputs a rotation angle signal indicating the angle of the rotation axis 201a. The control circuit 22 utilizes the rotation angle signal to generate the photon count distribution signal, and is able to measure the number of photons of from the measurement chamber 116 by utilizing the photon count distribution signal.

The display device 5 displays measurement values of photons. The display device 5 is a display panel such as a liquid crystal display panel or an organic EL panel, and displays measurement values of photons and/or information based on measurement values that is output from the control circuit 22, as well as past measurement values. Note that the display device 5 displays other information, e.g., methods of manipulating the sample analysis device 1, information to prompt an input for manipulation, for example.

Measurement values of photons may be transmitted to the outside of the sample analysis device 1 via the communication circuit 36. The communication circuit 36 may be a circuit which performs wired communication based on e.g. the Ethernet (registered trademark) standards, or a circuit which performs wireless communication based on e.g. the Wi-Fi (registered trademark) standards.

By executing the computer program stored in the internal memory 22a, the control circuit 22 realizes the aforementioned operation of the sample analysis device 1, and controls the drive circuit 20 to change the relative positions of the first magnet unit 16 and the sample analysis substrate 100 as will be described later.

Note that the memory 22a into which a computer program is loaded, e.g., a RAM storing a computer program, may be volatile or non-volatile. A volatile RAM is a RAM which in the absence of supplied power is unable to retain the information that is stored therein. For example, a dynamic random access memory (DRAM) is a typical volatile RAM. A non-volatile RAM is a RAM which is able to retain information without power being supplied thereto. For example, a magnetoresistive RAM (MRAM), a resistive random access memory (ReRAM), and a ferroelectric memory (FeRAM) are examples of non-volatile RAMs. A volatile RAM and a non-volatile RAM are both examples of non-transitory, computer-readable storage media. Moreover, a magnetic storage medium such as a hard disk, and an optical storage medium such as an optical disc are also examples of non-transitory, computer-readable storage media. That is, a computer program according to the present disclosure may be recorded on various non-transitory computer-readable media, excluding any medium such as the atmospheric air (transitory media) that allows a computer program to be propagated as a radiowave signal.

Next, the construction of the first magnet unit 16 and the operation of the sample analysis device 1 for changing the relative positions of the first magnet unit 16 and the sample analysis substrate 100 will be described.

FIG. 8 is an exploded perspective view of the first magnet unit 16. FIG. 9 is a plan view of the first magnet unit 16. As shown in FIG. 8 and FIG. 9, the first magnet unit 16 includes a magnet 40 and a case 42. The case 42 accommodates the magnet 40, fixing it within the case 42.

The magnet 40 is a magnet that is commonly used in immunoassay techniques based on a competitive assay that utilizes magnetism particles, for example. Specifically, a ferrite magnet, a neodymium magnet, or the like may be used as the magnet 40. In particular, a neodymium magnet is suitable as the magnet 40 because of having a strong magnetic force.

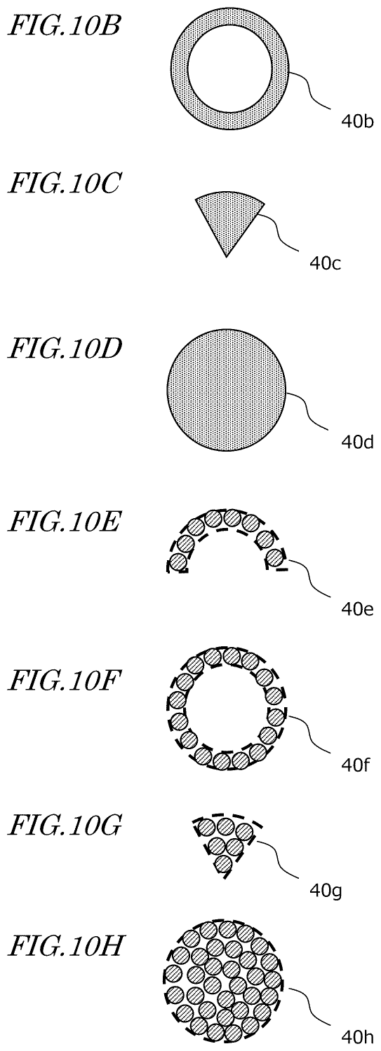

Although the magnet 40 has a semicircular-ring shape in FIG. 8 and FIG. 9, this is an example. Other shapes may also be adopted. FIG. 10A to FIG. 10D show example shapes for the magnet 40 that can be adopted in the present embodiment. FIG. 10A shows a semicircular-ring shape magnet 40a as described earlier. FIG. 10B shows a magnet 40b having a ring shape, i.e., a circular shape with an opening in the center. FIG. 10C shows a magnet 40c having a sector shape. FIG. 10D shows a magnet 40d having a circular shape. The shape of the case 42 may be adapted to the shape of any one of the magnets 40a to 40d that is adopted.

While FIG. 10A to FIG. 10D each illustrate an example shape for a single magnet, it is also possible to use a plurality of magnets. FIG. 10E to FIG. 10H illustrate examples in which a plurality of magnets are used to realize similar shapes to those of the magnets 40a to 40d shown in FIG. 10A to FIG. 10D. FIG. 10E shows a group of multiple magnets 40e that are arranged in a semicircular-ring shape. FIG. 10F shows a group of magnets 40f that are arranged in a ring shape, i.e., a circular shape with an opening in the center. FIG. 10G shows a group of magnets 40g that are arranged in a sector shape.

FIG. 10H shows a group of magnets 40h that are arranged in a circular shape. The shape of the case 42 may be adapted to the shape of any one of the group of magnets 40e to 40h that is adopted.

Although a single magnet exists or a single group of multiple magnets is kept together in FIG. 10A to FIG. 10H, a plurality of magnets that are distant from one another may instead be used. In that case, for example, a semicircular-ring shape and a sector shape may be combined. In the examples of FIG. 10A to FIG. 10H, the ring shape (semicircle or circle), the sector shape, and the circular shape do not need to be based on a perfect circle, but may each be a shape based on an ellipse. In the present embodiment, the magnet or the group of magnets may have a whole shape or a partial shape of a circle or a ring such that a sum of central angles of a circle(s) or an ellipse(s) is not less than 90 degrees and not more than 360 degrees.

Next, details of the mechanism and operation of driving the first magnet unit 16 will be described. The mechanism is provided within the housing 2 of the sample analysis device 1. Hereinafter, only the necessary component elements will be illustrated and described, while component elements which are not particularly needed, e.g., the housing 2 and the door 3, will be omitted from illustration and description.

FIG. 11 and FIG. 12 are a plan view and a side view showing a semicircular-ring shaped first magnet unit 16 having moved to above the circular sample analysis substrate 100, and the construction of the moving mechanism for the first magnet unit 16. First, the moving mechanism for the first magnet unit 16 will be described. As mentioned above, the number of magnets to be used for the first magnet unit 16 and its/their shape(s) may be arbitrary.

In the present embodiment, the first magnet unit 16 is located on an opposite side of the sample analysis substrate 100 from the turntable 10. However, the first magnet unit 16 may be located on the same side of the sample analysis substrate 100 as the turntable 10.

The first magnet unit 16 is driven by the first actuator 18. It is assumed that the first actuator 18 is an electric motor that undergoes rotational motion. A pinion gear 18a is attached to a shaft of the electric motor, and meshes with the first rack 44. Based on a command from the control circuit 22, the drive circuit 20 rotates the first actuator 18 clockwise or counterclockwise, or stops its rotation. As the first actuator 18 rotates clockwise, or rotates counterclockwise, the pinion gear 18a sends out the first rack 44 in the lower direction or the upper direction in the figure. Then, the first magnet unit 16 attached to the first rack 44 moves closer to the sample analysis substrate 100, or moves away from the sample analysis substrate 100.

The first actuator 18 moves the first magnet unit 16 along a direction that is perpendicular to the rotation axis 102 of the sample analysis substrate 100, i.e., a direction that is parallel to the circular surface of the sample analysis substrate 100. In order to achieve movement of the first magnet unit 16, a pair of guides 50 are provided in FIG. 11. For example, each guide 50 has a cross section with a substantially angular “U” shape, such that an upper face and a lower face of the first magnet unit 16 are sandwiched in its groove. As a result, movement of the sample analysis substrate 100 is restricted so as to occur exclusively along the longitudinal direction of the guides 50.

During a B/F separation for separating reacted substance from unreacted substance within the chamber, the first actuator 18 moves the first magnet unit to a position where the magnetic particles in the measurement chamber 116 are attracted by the first magnet unit 16. Specifically, the first actuator 18 moves the first magnet unit 16 to the position depicted in FIG. 11 and FIG. 12, and stabilizes it at that position.

The unreacted substance that has not been involved in the antigen-antibody reaction in the reaction chamber 114 is thereafter transferred to the measurement chamber 116 together with the reacted substance. Since a B/F separation is performed in order to remove the unreacted substance (non-magnetic component) existing in the measurement chamber 116, it is required that the magnetic force of the magnet(s) in the first magnet unit 16 effectively attracts the magnetic particles existing in the measurement chamber 116. Therefore, the radius size of the ring of the first magnet unit 16 is determined in accordance with the position of the measurement chamber 116 of the sample analysis substrate 100 when stabilized to that position. In other words, the radius size of the ring of the first magnet unit 16 is determined in accordance with the distance from the rotation axis 102 (center of rotation) of the sample analysis substrate 100 to the measurement chamber 116.

More specifically, the central position of the ring of the first magnet unit 16 regarding the radial direction is matched to the position in the measurement chamber 116 that is the farthest from the center of rotation of the sample analysis substrate 100. FIG. 11 shows two circles drawn with broken lines. The inner circle fits along the innermost periphery of the first magnet unit 16, and passes through the substantial central position of the measurement chamber 116 regarding the radial direction. On the other hand, the outer circle fits along the central position of the ring of the first magnet unit 16 regarding the radial direction, and passes through the outermost position of the measurement chamber 116 regarding the radial direction.

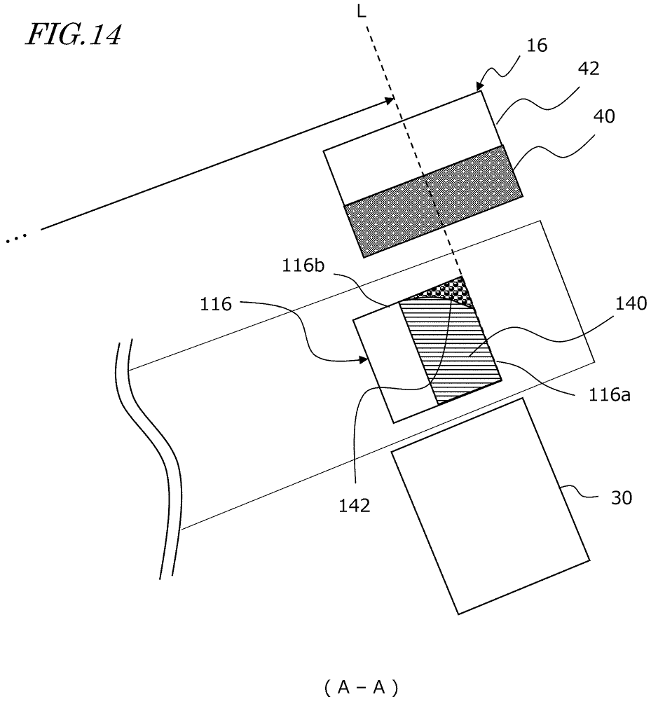

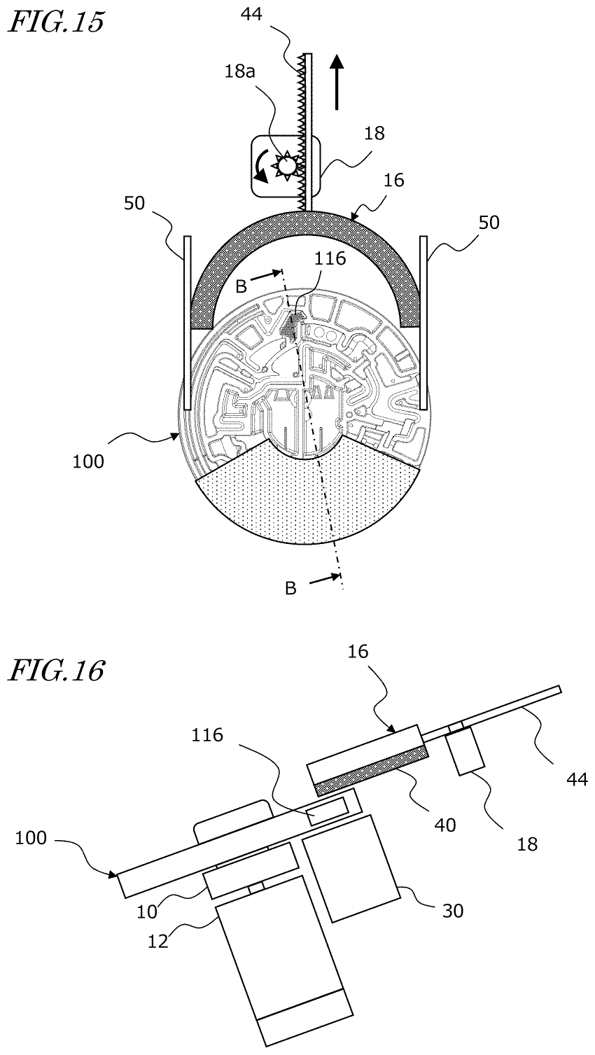

FIG. 13 shows a relationship between the position of the first magnet unit 16 and the position of the measurement chamber 116 after the sample analysis substrate 100 has been rotated by about 180°. FIG. 13 only shows the outer circle (broken line) in FIG. 11. Moreover, FIG. 14 shows an A-A cross section in FIG. 13. For ease of explanation, FIG. 14 shows enlarged a cross section near the measurement chamber 116.

As is particularly clear from FIG. 14, it will be appreciated that the central position L of the ring of the first magnet unit 16 regarding the radial direction matches the position 116a in the measurement chamber 116 that is the farthest from the center of rotation. Regarding the radial direction of the sample analysis substrate 100, magnetic particles 142 gather toward the position 116a in the measurement chamber 116 that is the farthest from the center of rotation, owing to the action of the centrifugal force during rotation of the sample analysis substrate 100. The magnetic particles 142 are the magnetic particles 302 contained in the composite 310, and the magnetic particles 302 having the primary antibody 304 immobilized to their surfaces. Note that the latter includes those magnetic particles 302 which have been produced from an antigen-antibody reaction between the primary antibody 304 and the antigen 306 and those magnetic particles 302 which have not.

On the other hand, regarding the direction of the rotation axis of the sample analysis substrate 100, the magnetic particles 142 stick to a position 116b in the measurement chamber 116 owing to the attractive force of the magnet 40 of the first magnet unit 16. In other words, the magnetic particles 142 can be effectively attracted. By appropriately rotating the sample analysis substrate 100 in this state, it is possible to transfer the reaction solution from the measurement chamber 116 to another chamber while leaving the magnetic particles 142 in the measurement chamber 116. Thereafter, while attracting the magnetic particles 142 with the magnetic force, a wash solution/substrate solution, for example, may be transferred to the measurement chamber 116 and discharged.

Moreover, as shown in FIG. 13, the length of the first magnet unit 16 along the circumferential direction is longer than the length of the measurement chamber 116 along the circumferential direction. This allows the attractive force to be applied to the entirety of the magnetic particles within the measurement chamber 116. Moreover, because the first magnet unit 16 has a whole shape or a partial shape of a circle or a ring, even with the sample analysis substrate 100 kept rotated, it is possible to prolong the duration of time in which the magnetic force from the first magnet unit 16 acts on the measurement chamber 116 during one turn of the sample analysis substrate 100, whereby a B/F separation based on the magnetic force can be better performed.

FIG. 15 and FIG. 16 are a plan view and a side view showing the first magnet unit 16 having been moved to a position retracted from above the sample analysis substrate 100, and the construction of the moving mechanism for the first magnet unit 16.

The first actuator 18 moves the first magnet unit 16 to a position at which the first magnet unit 16 and the sample analysis substrate 100 do not overlap as viewed in a direction that is parallel to the rotation axis of the sample analysis substrate 100.

Specifically, while the first magnet unit 16 is in the state shown in FIG. 13, the drive circuit 20 rotates the first actuator 18 counterclockwise, based on a command from the control circuit 22. Because of the counterclockwise rotation of the first actuator 18, the pinion gear 18a sends out the first rack 44 in the upper direction in the figure. Then, the first magnet unit 16 attached to the first rack 44 moves along a direction that is parallel to the circular surface of the sample analysis substrate 100, thus moving away from the sample analysis substrate 100.

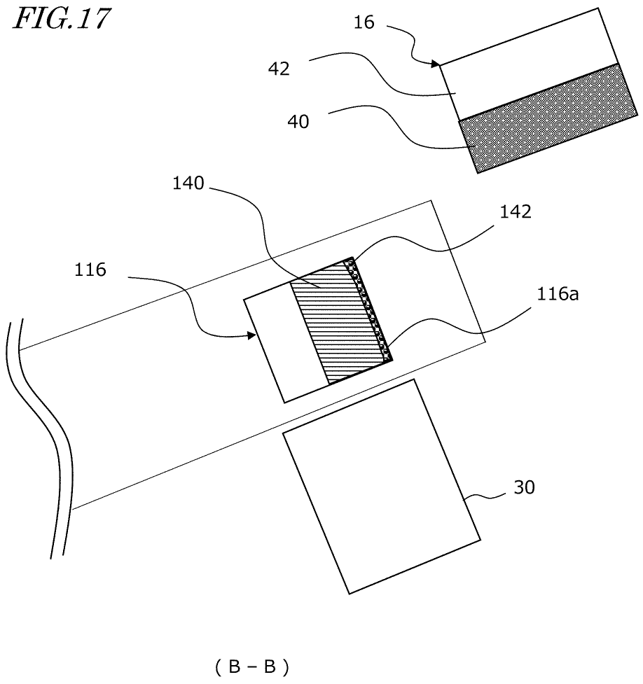

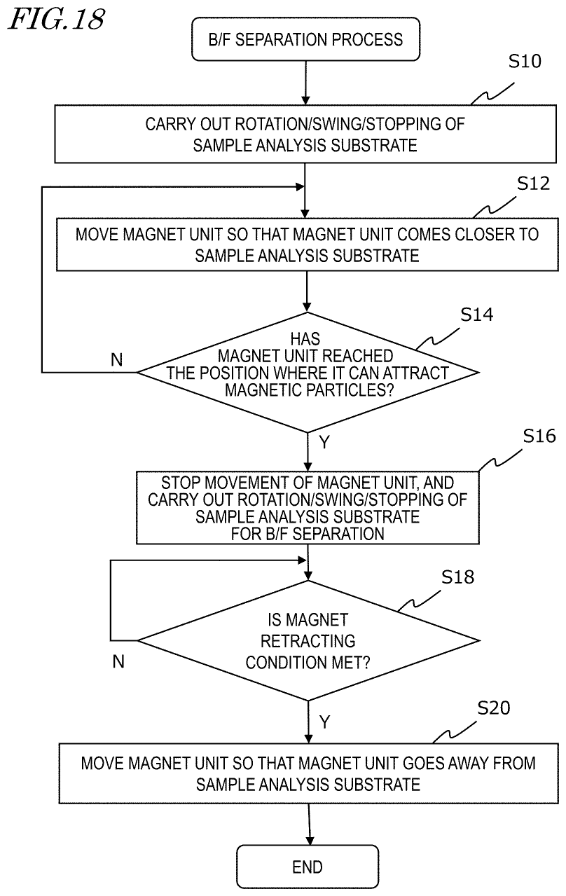

FIG. 17 shows a B-B cross section in FIG. 15. For ease of explanation, FIG. 17 shows enlarged a cross section near the measurement chamber 116.

As is clear from FIG. 17, as the first magnet unit 16 moves away from the sample analysis substrate 100, the magnetic force of the magnet 40 of the first magnet unit 16 becomes weaker. Consequently, the magnetic particles 142 are released from the attractive force of the magnet 40, and the magnetic particles 142 spread toward the position 116a in the measurement chamber 116 that is the farthest from the center of rotation. Through B/F separation, non-magnetic components containing uncaptured impurities are removed, thus resulting in a better washing effect on the reaction product, whereby a highly accurate analysis result can be obtained.

Thus, by moving the first magnet unit 16 to change the relative positions of the first magnet unit 16 and the sample analysis substrate 100, the magnetic particles can be effectively attracted; the reaction solution containing unreacted substance can be transferred from the measurement chamber 116; and the unreacted substance can be removed in another chamber.

FIG. 18 is a flowchart showing a procedure of processing by the control circuit 22 during the B/F separation process. The control circuit 22 executes a computer program that contains instructions for carrying out the processes described in the flowchart.

At step S10, the control circuit 22 controls operation of the motor 12 via the drive circuit 14, thereby carrying out rotation/swing/stopping of the sample analysis substrate 100. As a result, the control circuit 22 causes an antigen-antibody reaction in the reaction chamber 114, and transfers the reaction solution containing unreacted substance to the measurement chamber 116.

At step S12, the control circuit 22 controls operation of the first actuator 18 via the drive circuit 20, and moves the first magnet unit 16 so that the first magnet unit 16 comes closer to the sample analysis substrate 100.

At step S14, the control circuit 22 determines whether the first magnet unit 16 has reached the position where it can attract magnetic particles or not. Specifically, as shown in FIG. 14, the control circuit 22 determines whether the central position L of the ring of the first magnet unit 16 regarding the radial direction has matched the position 116a in the measurement chamber 116 that is the farthest from the center of rotation or not.

In the determination, the control circuit 22 may utilize an output of a sensor (not shown) to detect the position of the first magnet unit 16. Alternatively, in the case where the first actuator 18 is a stepping motor, the position of the first magnet unit 16 may be determined based on the number of driving pulses that have been transmitted to the first actuator 18. The amount by which the stepping motor is driven is in proportion to the number of driving pulses given. In other words, the amount of move of the first magnet unit 16 can be determined based on the number of driving pulses that have been transmitted to the first actuator 18. The position (fixed position) of the first magnet unit 16 immediately after the sample analysis substrate 100 was mounted is utilized as a reference. Assuming that N driving pulses are required until the central position L (FIG. 14) of the first magnet unit 16 being at the reference position matches the position 116a in the measurement chamber 116 (FIG. 14), the control circuit 22 may make the determination of step S14 by determining whether the number of driving pulses transmitted to the first actuator 18 has reached N or not.

If step S14 finds that the first magnet unit 16 has reached the position where it can attract magnetic particles, the process proceeds to step S16; it has not, the process returns to step S12.

At step S16, the control circuit 22 stops movement of the first magnet unit 16, and carries out rotation/swing/stopping of the sample analysis substrate 100 for B/F separation. Through this operation, the magnetic component containing magnetic particles that have been captured by the magnetic force of the first magnet unit 16 can be separated from non-magnetic components containing uncaptured impurities.

At step S18, the control circuit 22 determines whether a predetermined magnet retracting condition is satisfied or not. The “predetermined magnet retracting condition” may be that the operation of transferring a wash solution/substrate solution to the measurement chamber 116 and discharging it has been completed a predetermined number of times (i.e., B/F separation has been completed); the open-close detection switch 4 has detected opening of the door 3 during analysis of the sample; and so on, for example. The control circuit 22 continues the determination of step S18 until the predetermined magnet retracting condition is satisfied; once it is determined to be satisfied, the process proceeds to step S20.

At step S20, the control circuit 22 moves the first magnet unit 16 so that the first magnet unit 16 goes away from the sample analysis substrate 100.

Thus, the B/F separation process is finished.

Next, a modification of the sample analysis device 1 will be described. In FIG. 3, the sample analysis device 1 moves a single first magnet unit 16 in order to separate the composite 310 containing magnetic particles, or unreacted magnetic particles, from any unreacted substance other than magnetic particles. The sample analysis device 1 according to the modification includes a plurality of magnet units, and a moving mechanism for driving each of the plurality of magnet units.

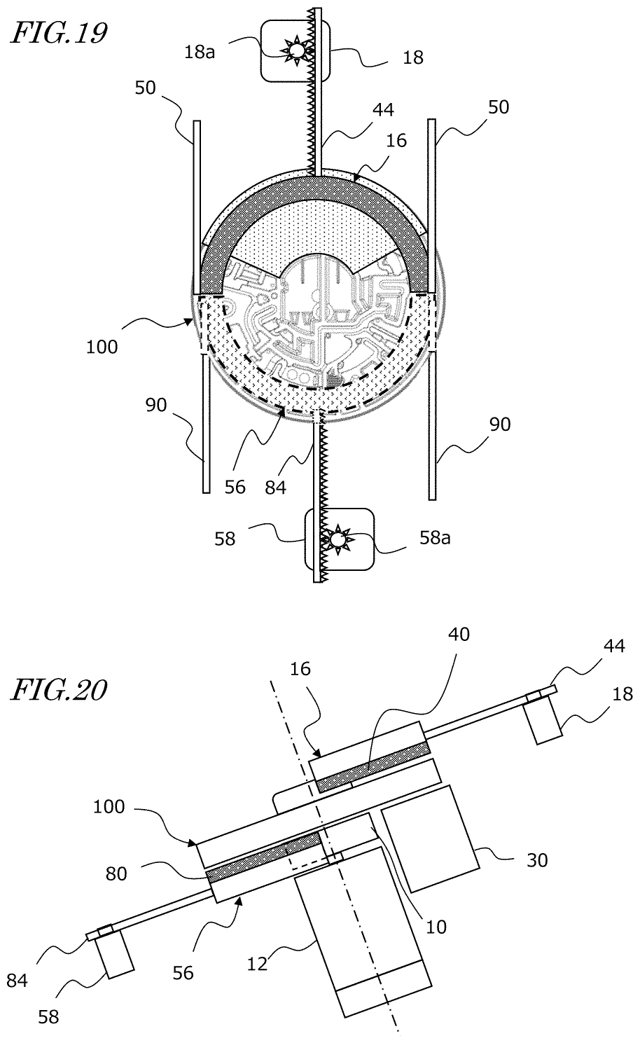

FIG. 19 and FIG. 20 are a plan view and a side view showing the construction of a semicircular-ring shaped first magnet unit 16 and second magnet unit 56 and moving mechanisms for moving the first magnet unit 16 and the second magnet unit 56. The relationship between the first magnet unit 16 and the first actuator 18 for driving the first magnet unit 16 is as has been described earlier, and therefore its description is omitted.

In the example shown in FIG. 19, the second magnet unit 56 is identical in shape to the first magnet unit 16. However, as does the first magnet unit 16, the second magnet unit 56 may also include a single magnet having a whole shape or a partial shape of a circle or a ring, or a plurality of magnets arranged along the shape, as illustrated in FIG. 10A to FIG. 10H.

As can be understood from FIG. 20, the second magnet unit 56 is located on the same side of the sample analysis substrate 100 as the turntable 10. The second magnet unit 56 is driven by a second actuator 58. In this modification, the second actuator 58 is an electric motor that undergoes rotational motion. The second actuator 58 may be a stepping motor or a linear motor, for example. A drive circuit (not shown) for driving the second actuator 58 is also separately provided, which is controlled by the control circuit 22.

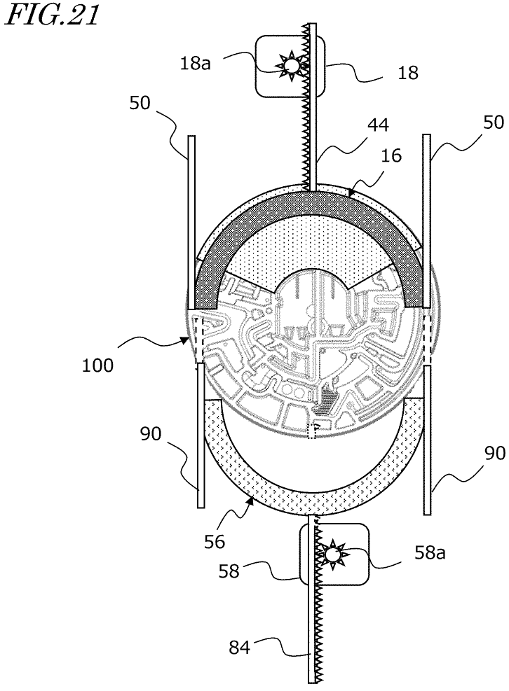

A pinion gear 58a (FIG. 19) is attached to a shaft of the second actuator 58, and meshes with the second rack 84. Based on a command from the control circuit 22, the drive circuit rotates the second actuator 58 clockwise or counterclockwise, or stops its rotation. As the second actuator 58 rotates clockwise, or rotates counterclockwise, the pinion gear 58a sends out the second rack 84 in the upper direction or the lower direction in the figure. Then, the second magnet unit 56 attached to the second rack 84 moves closer to the sample analysis substrate 100, or moves away from the sample analysis substrate 100. FIG. 21 shows the second magnet unit 56 having moved away from the sample analysis substrate 100.

The second actuator 58 moves the second magnet unit 56 along a direction that is perpendicular to the rotation axis 102 of the sample analysis substrate 100, or a direction that is parallel to the circular surface of the sample analysis substrate 100. In order to achieve movement of the second magnet unit 56, a pair of guides 90 are provided in FIG. 21. For example, each guide 90 also has a cross section with a substantially angular “U” shape, such that an upper face and a lower face of the second magnet unit 56 are sandwiched in its groove. As a result, movement of the sample analysis substrate 100 is restricted so as to occur exclusively along the longitudinal direction of the guides 90.

According to this modification, in addition to moving the first magnet unit 16 closer to or away from one side of the sample analysis substrate 100, it is also possible to move the second magnet unit 56 closer to or away from the other side of the sample analysis substrate 100. This may allow the magnetic particles to be kept adsorbed to a desired side of the sample analysis substrate 100.

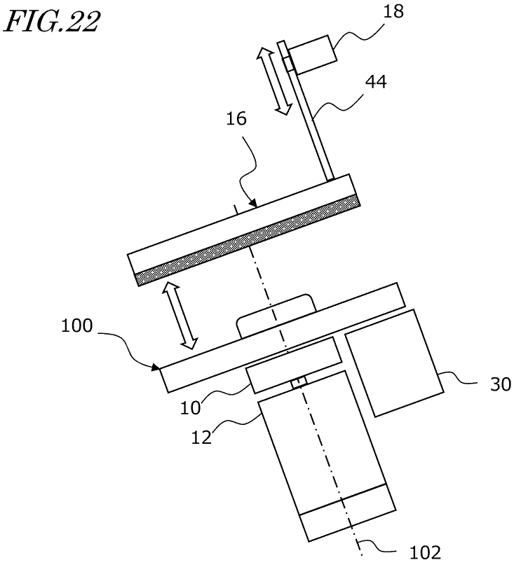

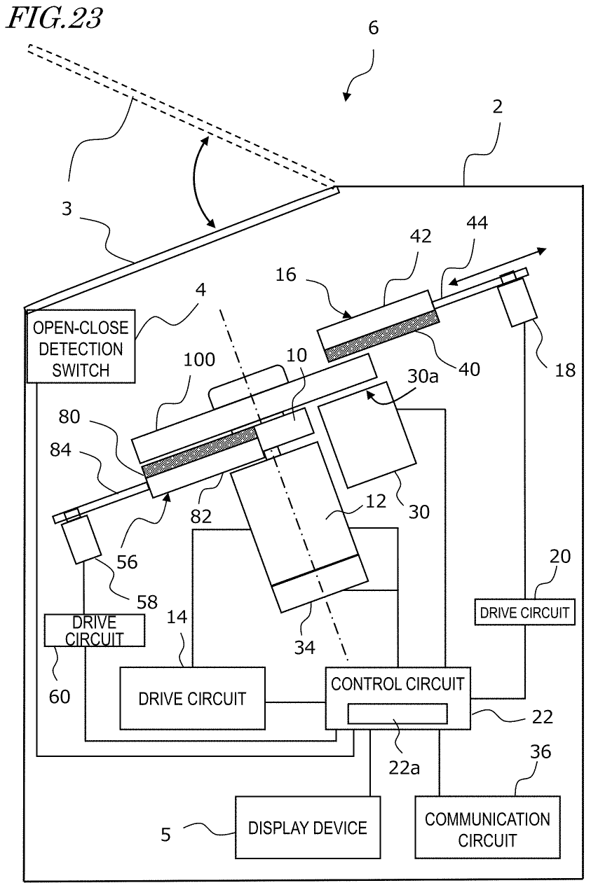

FIG. 22 is a side view for describing a modification concerning the moving directions of the first magnet unit 16. In this modification, the first magnet unit 16 is driven in a direction that is parallel to the rotation axis 102 of the sample analysis substrate 100, whereby the relative positions of the first magnet unit 16 and the sample analysis substrate 100 are changed. Therefore, the orientations in which the first actuator 18 and the first rack 44 are attached are different from those in the example construction of FIG. 12. Aspects other than the orientations are the same as in the example construction of FIG. 12. Therefore, further description will be omitted.

In the example of FIG. 22, there exists a single first magnet unit 16. However, another first magnet unit and first actuator may be provided, similarly to the second magnet unit 56 described with reference to FIG. 19 to FIG. 21, for movement along a direction that is parallel to the rotation axis 102.

The description of the above embodiment and its modifications has illustrated implementations where a pinion gear and a rack are utilized in order to drive each first magnet unit. However, such implementations are only examples, and other mechanisms can also be used. For example, the first magnet unit and the motor may be mechanically connected, and the position of the first magnet unit may be changed with the rotational position of the motor, thus realizing retraction from the sample analysis substrate 100 and approach to the sample analysis substrate 100. The structure for moving the first magnet unit may as a whole be referred to as the “magnet moving mechanism”.

### Second Embodiment

A second embodiment of a sample analysis device according to the present embodiment will be described. In the aforementioned measurement method utilizing magnetic particles, in order to more accurately measure the concentration of the antigen 306 in a sample, it is desirable to produce a composite 310 in which as much antigen 306 in the sample is bound to the magnetic-particle-immobilized antibody 305 and the labeled antibody 308 as possible. For this, it is desirable to allow as much antigen-antibody reaction to occur between the antigen 306 and the magnetic-particle-immobilized antibody 305 and labeled antibody 308 as possible.

Conventional sample analysis devices have agitated a sample-containing solution through swinging operations of consecutively reversing the rotating direction of the sample analysis substrate. However, conventional methods have much room for improving measurement accuracy.

For example, coagulation between the magnetic particles and the unreacted sample (blood, etc.) may occur within the solution. Because such coagulation cannot be eliminated through swinging operations, there have been cases where the solution did not really receive sufficient agitation.

In the B/F separation step, the magnetic particles are captured by using a magnet(s), and the reaction solution is discharged in this state. Thereafter, a wash solution is dispensed into a chamber. At this time, even if the sample analysis device swings the sample analysis substrate for washing purposes, the unreacted sample may have been captured between the coagulated magnetic particles that were attracted to the magnet(s). This has led to cases where the solution washing was not really sufficiently performed.

In the sample analysis device according to the present embodiment, the first magnet unit and the second magnet unit are disposed on, respectively, a first face of the sample analysis substrate and a second face that is opposite to the first face. During agitation of the liquid sample in a chamber in the B/F separation step, for example, the sample analysis device alternately moves the first magnet unit or the second magnet unit to a position where the magnetic particles in the chamber are attracted by the magnet unit. When the first magnet unit moves so as to come closer to the sample analysis substrate, the magnetic particles are attracted toward the first face; when the second magnet unit moves so as to come closer to the sample analysis substrate, the magnetic particles are attracted toward the second face. Since this allows the magnetic particles to be agitated, an improved washing effect can be provided, and thus an enhanced measurement accuracy for a specific component in a sample can be obtained.

FIG. 23 is a block diagram showing an example hardware configuration of a sample analysis device 6 according to the present embodiment. The sample analysis device 6 according to the present embodiment differs from the sample analysis device 1 of the first embodiment in that a drive circuit 60, a second magnet unit 56, and a second actuator 58 are further included.

As does the first magnet unit 16, the second magnet unit 56 includes one or more magnets, and with the one or more magnets, generates a force (magnetic force) to attract the magnetic particles. The second magnet unit 56 has a “whole or partial” shape of “a circle or a ring”. A “whole or partial” shape of “a circle or a ring” is achieved by the shape of a single magnet or an arrangement of a plurality of magnets. The specific construction of the second magnet unit 56 will be described later. To the second magnet unit 56, a second rack 84 having teeth thereon is attached.

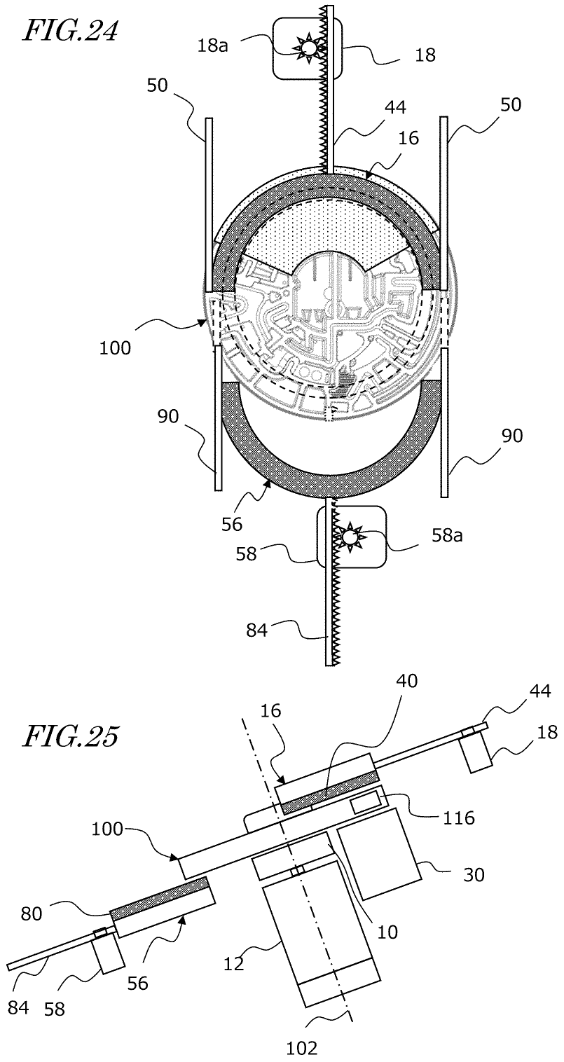

The second actuator 58 moves the second magnet unit 56 by moving the second rack 84 along the longitudinal direction, thereby changing the relative positions of the second magnet unit 56 and the sample analysis substrate 100. The operation of the second actuator 58 is controlled by the drive circuit 60. An example of the second actuator 58 is an electric motor that undergoes rotational motion. The second actuator 58 may be a stepping motor or a linear motor, for example. Details of the construction and operation regarding the second actuator 58 will also be described later with reference to FIG. 24, FIG. 25, and so on.

By executing the computer program stored in the internal memory 22a, the control circuit 22 realizes the aforementioned operation of the sample analysis device 6, and controls the drive circuit 20 to change the relative positions of the first magnet unit 16 and the sample analysis substrate 100, and the relative positions of the second magnet unit 56 and the sample analysis substrate 100, as will be described later.

Next, the construction of the first magnet unit 16 and the second magnet unit 56 and the operation of the sample analysis device 6 for changing the relative positions of each magnet unit and the sample analysis substrate 100 will be described.

The second magnet unit 56 and the second actuator 58 for driving the second magnet unit are configured similarly to the first magnet unit 16 and the first actuator 18. However, the first magnet unit 16 and the second magnet unit 56 are independent of each other, and it is not necessary to adopt the same construction for them. For example, the shape of the magnet 80 of the second magnet unit 56 may be different from the shape of the magnet 40 of the first magnet unit 16 described below. This is also true of the shape of a case 82 that accommodates the magnet 80.

Next, details of the mechanism and operation of driving each magnet unit will be described. The mechanism is provided within the housing 2 of the sample analysis device 6. Hereinafter, only the necessary component elements will be illustrated and described, while component elements which are not particularly needed, e.g., the housing 2 and the door 3, will be omitted from illustration and description.

FIG. 24 and FIG. 25 illustrate an example relative positioning between the first magnet unit 16, the second magnet unit 56, and the sample analysis substrate 100. In the present embodiment, the first magnet unit 16 is located on an opposite side of the sample analysis substrate 100 from the turntable 10, whereas the second magnet unit 56 is located on the same side of the sample analysis substrate 100 as the turntable 10. In the present embodiment, during a B/F separation and/or a luminescence measurement, as viewed from a direction that is parallel to the rotation axis 102 of the sample analysis substrate 100, the first magnet unit 16 and the second magnet unit 56 are driven so as not to overlap the sample analysis substrate 100 at the same time. Therefore, the control circuit 22 controls operation of the first magnet unit 16 and the second magnet unit 56 so as to result in either: only the first magnet unit 16 overlapping the sample analysis substrate 100; only the second magnet unit 56 overlapping the sample analysis substrate 100; or neither the first magnet unit 16 nor the second magnet unit 56 overlapping the sample analysis substrate 100. In the example of FIG. 24 and FIG. 25, only the first magnet unit 16 overlaps the sample analysis substrate 100, whereas the second magnet unit 56 has been retracted to a position not overlapping the sample analysis substrate 100.

As mentioned above, the number of magnets to be used for each of the first magnet unit 16 and the second magnet unit 56 and its/their shape(s) may be arbitrary, and may be independently determined.

Hereinafter, details of the method of driving of the first magnet unit 16 will be described.

Similarly to the first embodiment, the first magnet unit 16 is driven by the first actuator 18. It is assumed that the first actuator 18 is an electric motor that undergoes rotational motion. A pinion gear 18a is attached to a shaft of the electric motor, and meshes with the first rack 44. Based on a command from the control circuit 22, the drive circuit 20 rotates the first actuator 18 clockwise or counterclockwise, or stops its rotation. As the first actuator 18 rotates clockwise, or rotates counterclockwise, the pinion gear 18a sends out the first rack 44 in the lower direction or the upper direction in the figure. Then, the first magnet unit 16 attached to the first rack 44 moves closer to the sample analysis substrate 100, or moves away from the sample analysis substrate 100.

The first actuator 18 moves the first magnet unit 16 along a direction that is perpendicular to the rotation axis 102 of the sample analysis substrate 100, i.e., a direction that is parallel to the circular surface of the sample analysis substrate 100. In order to achieve movement of the first magnet unit 16, a pair of guides 50 are provided in FIG. 24. For example, each guide 50 has a cross section with a substantially angular “U” shape, such that an upper face and a lower face of the first magnet unit 16 are sandwiched in its groove. As a result, movement of the sample analysis substrate 100 is restricted so as to occur exclusively along the longitudinal direction of the guides 50.

During a B/F separation for separating reacted substance from unreacted substance within the chamber, the first actuator 18 moves the magnet unit to a position where the magnetic particles in the measurement chamber 116 are attracted by the first magnet unit 16. Specifically, the first actuator 18 moves the first magnet unit 16 to the position depicted in FIG. 24 and FIG. 25, and stabilizes it at that position.

The unreacted substance that has not been involved in the antigen-antibody reaction in the reaction chamber 114 is thereafter transferred to the measurement chamber 116 together with the reacted substance. Since a B/F separation is performed in order to remove the unreacted substance (non-magnetic component) existing in the measurement chamber 116, it is required that the magnetic force of the magnet(s) in the first magnet unit 16 effectively attracts the magnetic particles existing in the measurement chamber 116. Therefore, the radius size of the ring of the first magnet unit 16 is determined in accordance with the position of the measurement chamber 116 of the sample analysis substrate 100 when stabilized to that position. In other words, the radius size of the ring of the first magnet unit 16 is determined in accordance with the distance from the rotation axis 102 (center of rotation) of the sample analysis substrate 100 to the measurement chamber 116.

More specifically, the central position of the ring of the first magnet unit 16 regarding the radial direction is matched to the position in the measurement chamber 116 that is the farthest from the center of rotation of the sample analysis substrate 100. FIG. 24 shows two circles drawn with broken lines. The inner circle fits along the innermost periphery of the first magnet unit 16, and passes through the substantial central position of the measurement chamber 116 regarding the radial direction. On the other hand, the outer circle fits along the central position of the ring of the first magnet unit 16 regarding the radial direction, and passes through the outermost position of the measurement chamber 116 regarding the radial direction.

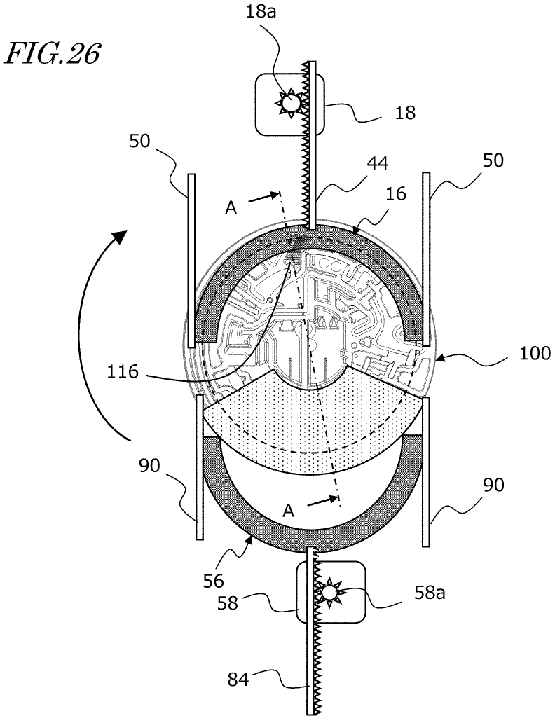

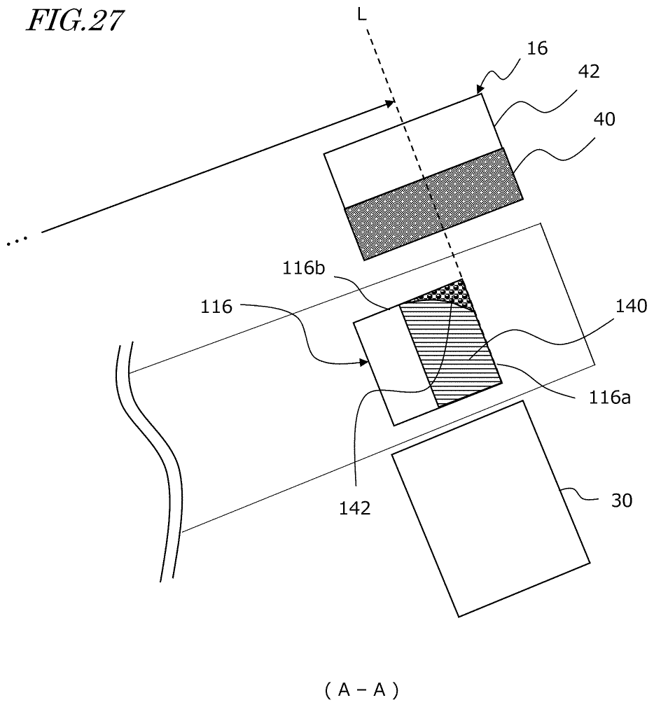

FIG. 26 shows a relationship between the position of the first magnet unit 16 and the position of the measurement chamber 116 after the sample analysis substrate 100 has been rotated by about 180° from the state shown in FIG. 24. FIG. 26 only shows the outer circle (broken line) in FIG. 24. Moreover, FIG. 27 shows an A-A cross section in FIG. 26. For ease of explanation, FIG. 27 shows enlarged a cross section near the measurement chamber 116.

As is particularly clear from FIG. 27, it will be appreciated that the central position L of the ring of the first magnet unit 16 regarding the radial direction matches the position 116a in the measurement chamber 116 that is the farthest from the center of rotation. Regarding the radial direction of the sample analysis substrate 100, magnetic particles 142 gather toward the position 116a in the measurement chamber 116 that is the farthest from the center of rotation, owing to the action of the centrifugal force during rotation of the sample analysis substrate 100. The magnetic particles 142 are the magnetic particles 302 contained in the composite 310, and the magnetic particles 302 having the primary antibody 304 immobilized to their surfaces. Note that the latter includes those magnetic particles which have been produced from an antigen-antibody reaction between the primary antibody 304 and the antigen 306 and those magnetic particles which have not.

On the other hand, regarding the direction of the rotation axis of the sample analysis substrate 100, the magnetic particles 142 stick to a position 116b in the measurement chamber 116 owing to the attractive force of the magnet 40 of the first magnet unit 16. In other words, the magnetic particles 142 can be effectively attracted. By appropriately rotating the sample analysis substrate 100 in this state, it is possible to transfer the reaction solution from the measurement chamber 116 to another chamber while leaving the magnetic particles 142 in the measurement chamber 116. Thereafter, while attracting the magnetic particles 142 with the magnetic force, a wash solution/substrate solution, for example, may be transferred to the measurement chamber 116 and discharged.

Moreover, as shown in FIG. 26, the length of the first magnet unit 16 along the circumferential direction is longer than the length of the measurement chamber 116 along the circumferential direction. This allows the attractive force to be applied to the entirety of the magnetic particles within the measurement chamber 116.

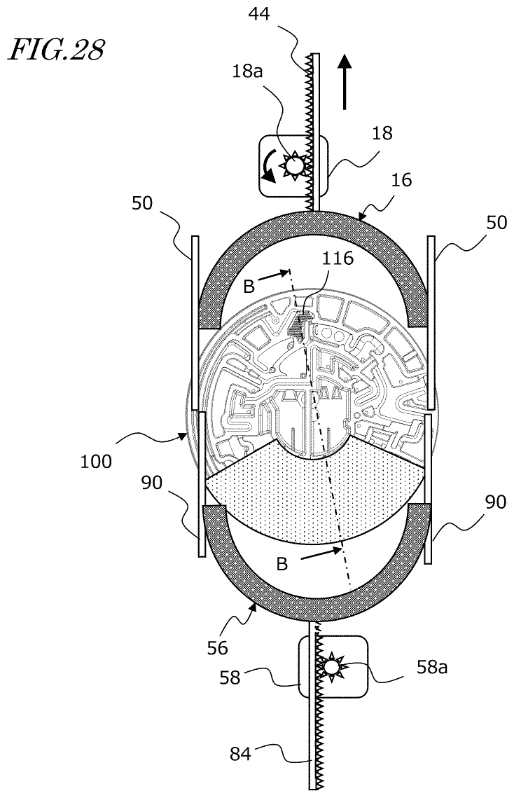

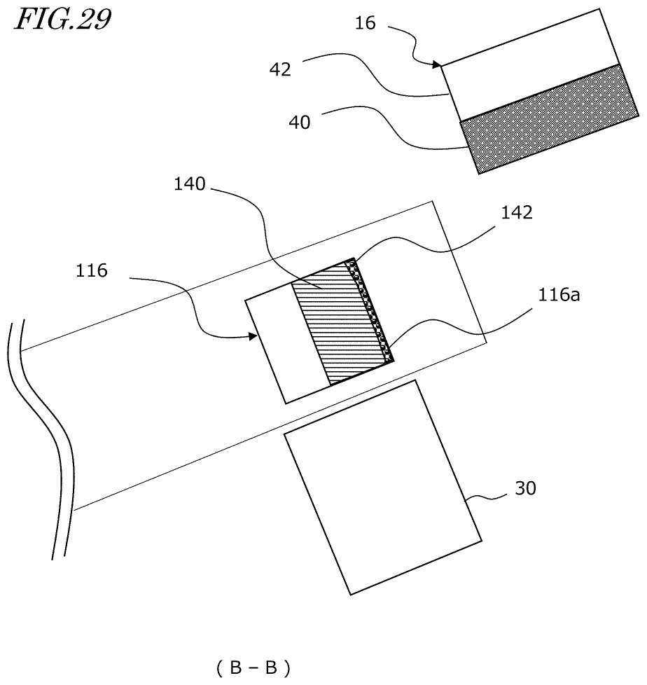

FIG. 28 and FIG. 29 are a plan view and a side view showing the first magnet unit 16 having been moved to a position retracted from above the sample analysis substrate 100, and the construction of the moving mechanism for the first magnet unit 16. The position of the second magnet unit 56 remains the same.

The first actuator 18 moves the first magnet unit 16 to a position at which the first magnet unit 16 and the sample analysis substrate 100 do not overlap as viewed in a direction that is parallel to the rotation axis of the sample analysis substrate 100.

Specifically, while in the state shown in FIG. 26, the drive circuit 20 rotates the first actuator 18 counterclockwise, based on a command from the control circuit 22. Because of the counterclockwise rotation of the first actuator 18, the pinion gear 18a sends out the first rack 44 in the upper direction in the figure. Then, the first magnet unit 16 attached to the first rack 44 moves along a direction that is parallel to the circular surface of the sample analysis substrate 100, thus going away from the sample analysis substrate 100.

FIG. 29 shows a B-B cross section in FIG. 28. For ease of explanation, FIG. 29 shows enlarged a cross section near the measurement chamber 116.

As is clear from FIG. 29, as the first magnet unit 16 moves away from the sample analysis substrate 100, the magnetic force of the magnet 40 of the first magnet unit 16 becomes weaker. Consequently, the magnetic particles 142 are released from the attractive force of the magnet 40, and the magnetic particles 142 spread toward the position 116a in the measurement chamber 116 that is the farthest from the center of rotation. Through B/F separation, non-magnetic components containing uncaptured impurities are removed, thus resulting in a better washing effect on the reaction product, whereby a highly accurate analysis result can be obtained.

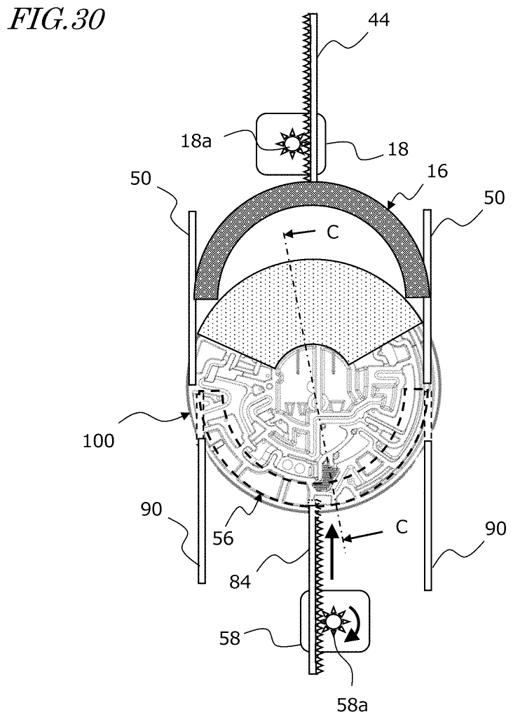

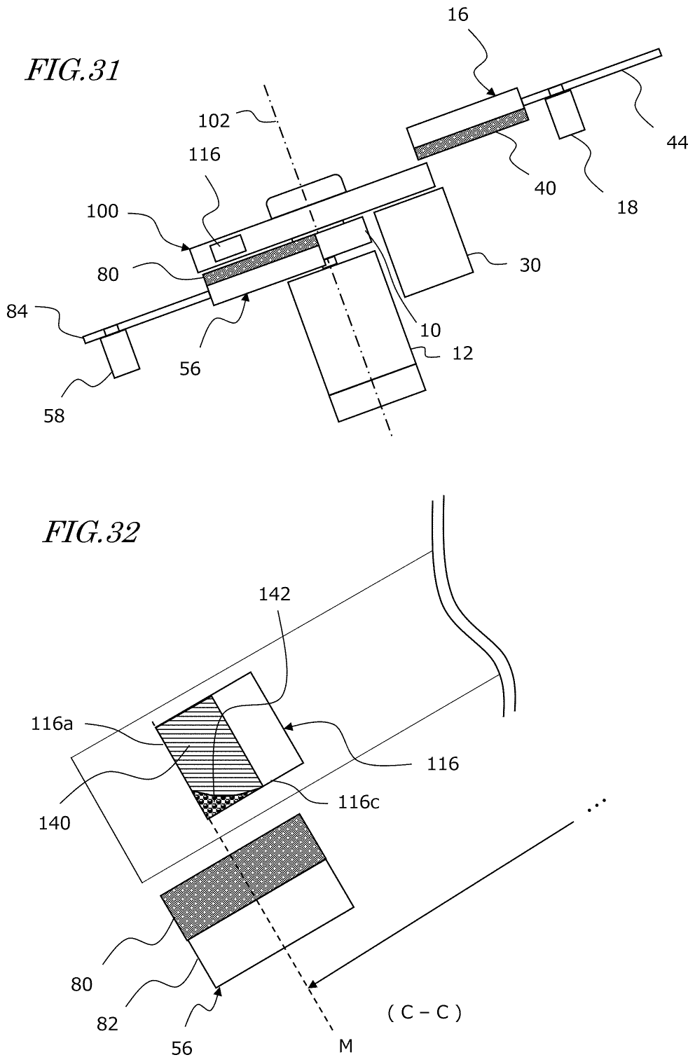

FIG. 30 and FIG. 31 are a plan view and a side view showing the second magnet unit 56 having moved to a position overlapping the sample analysis substrate 100 and the construction of the moving mechanism for the second magnet unit 56. The first magnet unit 16 remains at the position shown in FIG. 28.

The second actuator 58 moves the second magnet unit 56 to a position at which the second magnet unit 56 and the sample analysis substrate 100 overlap as viewed in a direction that is parallel to the rotation axis 102 of the sample analysis substrate 100.

Specifically, in the state shown in FIG. 28, the drive circuit 60 rotates the second actuator 58 clockwise, based on a command from the control circuit 22. Because of the clockwise rotation of the second actuator 58, the pinion gear 58a sends out the second rack 84 in the upper direction in the figure. Then, the second magnet unit 56 attached to the second rack 84 moves along a direction that is parallel to the circular surface of the sample analysis substrate 100, thus coming closer to the sample analysis substrate 100. Once the second magnet unit 56 reaches the position shown in FIG. 31, the second actuator 58 stops rotation. As a result, the second magnet unit 56 stops at the position shown in FIG. 31, and is stabilized at that position.

FIG. 32 shows a C-C cross section in FIG. 30. For ease of explanation, FIG. 32 shows enlarged a cross section near the measurement chamber 116.

The central position M of the ring of the second magnet unit 56 regarding the radial direction matches the aforementioned position 116a in the measurement chamber 116 that is the farthest from the center of rotation. Regarding the radial direction of the sample analysis substrate 100, the magnetic particles 142 gather at the position 116a in the measurement chamber 116 that is the farthest from the center of rotation, owing to the action of the centrifugal force during rotation of the sample analysis substrate 100.

Note that the central position L of the ring of the first magnet unit 16 regarding the radial direction (FIG. 27) and the central position M of the ring of the second magnet unit 56 regarding the radial direction both match the position 116a in the measurement chamber 116. Therefore, the distance from the rotation axis 102 to the central position L and the distance from the rotation axis 102 to the central position M are equal.

Thereafter, as necessary, retraction of the second magnet unit 56 and movement of the first magnet unit 16 to above the sample analysis substrate 100, as well as retraction of the first magnet unit 16 and movement of the second magnet unit 56 to below the sample analysis substrate 100, are effected. The first actuator 18 and the second actuator 58 alternately move the first magnet unit 16 and the second magnet unit 56 so that the magnetic particles 142 in the measurement chamber 116 come to a position where the magnetic particles 142 in the measurement chamber 116 are attracted by the first magnet unit 16 or the second magnet unit 56. The magnetic particles 142 transition between the attracted state shown in FIG. 27 and the released state shown in FIG. 29, and between the released state shown in FIG. 29 and the attracted state shown in FIG. 32. Through repetitions of attraction and release of the magnetic particles 142, the solution in the measurement chamber 116 is agitated. With the agitation, even if any unreacted sample has been captured between the coagulated magnetic particles 142, the unreacted sample is more likely to be released from the coagulation of magnetic particles 142. This realizes a further promotion of antigen-antibody reaction and/or washing of the solution.

Regarding the first magnet unit 16 and the second magnet unit 56 described above, the period with which to “alternately” effect their approach to the sample analysis substrate 100 and retraction from the sample analysis substrate 100 may be determined by considering the moving velocity of the magnetic particles 142, for example. Assume that a movement of the measurement chamber 116 of the sample analysis substrate 100 from the position 116b to the position 116c is known to take about 5 seconds, because of the components and viscosity of the solution, the rotational speed of the sample analysis substrate 100, and the like. Then, not less than about 10 seconds will be required for the magnetic particles 142 in the measurement chamber 116 to move from the position 116b shown in FIG. 27, through a released state (FIG. 29), to the position 116c shown in FIG. 32, and further return to the position 116b shown in FIG. 27 in the reverse order. Therefore, one cycle from beginning approach to the sample analysis substrate 100, through retraction, until returning to the same position may be set to 10 seconds. One skilled in the art shall be able to determine the moving velocity and acceleration from the beginning of the movement until stopping. For example, the acceleration immediately after beginning a retraction may be maximized so that one of the magnet units will promptly release the magnetic particles 142. Also, the acceleration immediately before coming to a stop during an approach to the sample analysis substrate 100 may be minimized so that the other magnet unit will promptly attract the magnetic particles 142.

Thus, by moving the first magnet unit 16 and the second magnet unit 56 so as to change the relative positions of the first magnet unit 16 and the sample analysis substrate 100, and to change the relative positions of the second magnet unit 56 and the sample analysis substrate 100, the magnetic particles are effectively attracted and released.

The aforementioned process can be performed in any kind of step so long as there are magnetic particles in the chamber. For example, the aforementioned process may be performed in a step of causing an antigen-antibody reaction by using a sample and dried reagents, or performed in a B/F separation step after the antigen-antibody reaction has been effected. The measurement accuracy for a specific component in a sample will be highest when the aforementioned process is performed in all such exemplified steps. However, even when the aforementioned process is performed only in at least one of those steps, the resulting measurement accuracy will be enhanced over the case where the solution is agitated only through swings of the sample analysis substrate 100.

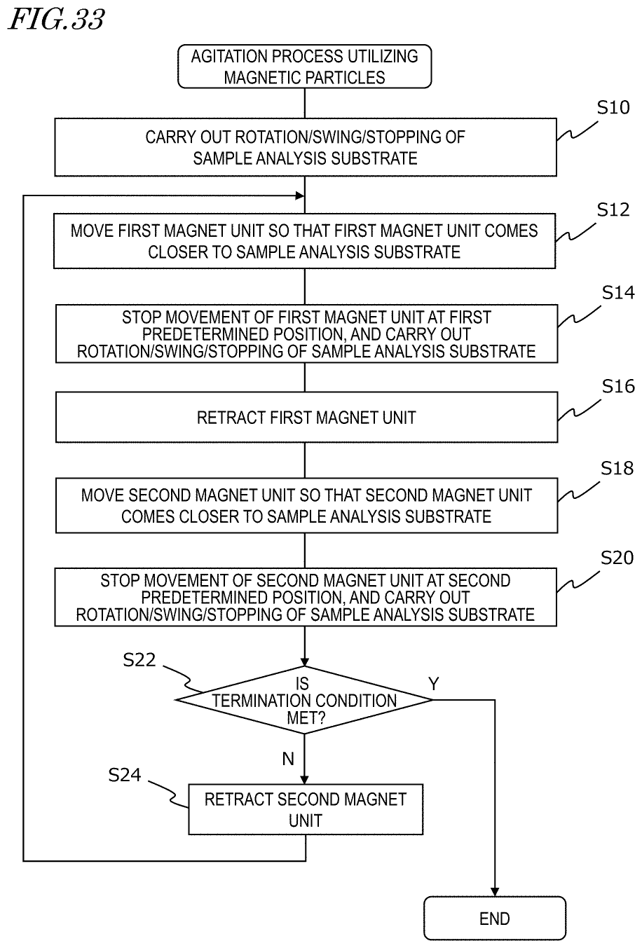

FIG. 33 is a flowchart showing a procedure of processing by the control circuit 22 of carrying out an agitation process utilizing magnetic particles. The control circuit 22 executes a computer program that contains instructions for carrying out the processes described in the flowchart. It is assumed that the first magnet unit 16 and the second magnet unit 56 have been retracted to the position shown in FIG. 28 before performing the process shown in FIG. 33. For example, a point immediately after the sample analysis substrate 100 has been mounted to the sample analysis device 6 and a sample has been apply dropwise may be envisaged.

At step S10, the control circuit 22 carries out rotation/swing/stopping of the sample analysis substrate 100.

At step S12, the control circuit 22 moves the first magnet unit 16 so that the first magnet unit 16 comes closer to the sample analysis substrate 100.

At step S14, the control circuit 22 stops movement of the first magnet unit 16 at a first predetermined position, and carries out rotation/swing/stopping of the sample analysis substrate 100. The “first predetermined position” is the position of the first magnet unit 16 when the central position L of the ring of the first magnet unit 16 regarding the radial direction has reached the position 116a in the measurement chamber 116 that is the farthest from the center of rotation. At this time, the control circuit 22 may perform a swing of the sample analysis substrate 100.

Then, at step S16, the control circuit 22 retracts the first magnet unit 16.

At step S18, the control circuit 22 then moves the second magnet unit 56 so that the second magnet unit 56 comes closer to the sample analysis substrate 100.

At step S20, the control circuit 22 stops movement of the second magnet unit 56 at a second predetermined position, and carries out rotation/swing/stopping of the sample analysis substrate 100. The “second predetermined position” is the position of the second magnet unit 56 shown in FIG. 32; that is, it is the position of the second magnet unit 56 when the central position M of the ring of the second magnet unit 56 regarding the radial direction has reached the position 116a in the measurement chamber 116 that is the farthest from the center of rotation. At this time, the control circuit 22 may perform a swing of the sample analysis substrate 100.

At step S22, the control circuit 22 determines whether a termination condition is satisfied. The “termination condition” may be any of the following, for example: an operation of alternately moving the first magnet unit 16 and the second magnet unit 56 has been finished a predetermined number of times (i.e., a predetermined number of times of agitation have been finished) in order to mix the sample with dried reagents to cause an antigen-antibody reaction; a predetermined time has elapsed; the operation of transferring a wash solution/substrate solution to the measurement chamber 116 and discharging it has been completed a predetermined number of times (i.e., B/F separation has been completed); the open-close detection switch 4 has detected opening of the door 3 during analysis of the sample; and so on. If it is satisfied, the process ends; if it is not satisfied, the process proceeds to step S24.

At step S24, the control circuit 22 retracts the second magnet unit 56. Thereafter, the process returns to step S12, and the process of step S12 and onwards is repeated.

Thus, agitation utilizing movement of magnetic particles is finished.

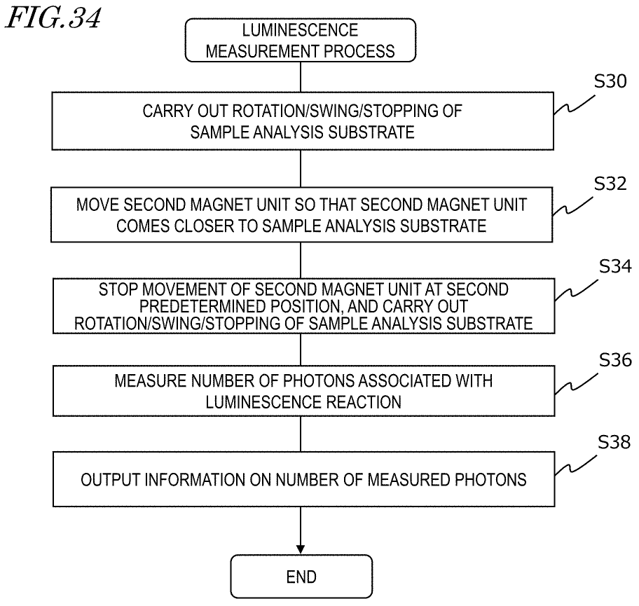

FIG. 34 is a flowchart showing a procedure of processing by the control circuit 22 carrying out a luminescence measurement process. Similarly to the earlier example of FIG. 33, the control circuit 22 executes a computer program that contains instructions for carrying out the processes described in the flowchart. Note that, before performing the process shown in FIG. 34, washing of the measurement chamber 116 has been completed and the first magnet unit 16 and the second magnet unit 56 has been again retracted to the position shown in FIG. 28.

At step S30, the control circuit 22 carries out rotation/swing/stopping of the sample analysis substrate 100.

At step S32, the control circuit 22 moves the second magnet unit 56 so that the second magnet unit 56 comes closer to the sample analysis substrate 100.

At step S34, the control circuit 22 stops movement of the second magnet unit 56 at a second predetermined position, and carries out rotation/swing/stopping of the sample analysis substrate 100. The second predetermined position is the same as that described regarding step S20 of FIG. 33.

At step S36, the control circuit 22 measures the number of photons associated with luminescence reaction.

At step S38, the control circuit 22 outputs (displays) information on the number of measured photons on the display device 5, for example.

Steps S34 and S36 will be more specifically described. As shown in FIG. 31, the second magnet unit 56 is located on the same side of the sample analysis substrate 100 as the photodetector 30. When the sample analysis substrate 100 rotates with the magnetic particles 142 being attracted by the second magnet unit 56, the magnetic particles 142 passes through a position that is the closest to the photodetector 30. Since the luminescence center when luminescence reaction occurs is near the magnetic particles 142, bringing the luminescence center closer to the photodetector 30 can increase the amount of light received by the photodetector 30. In other words, the measurement accuracy of the number of photons associated with luminescence reaction can be improved. As a result of this, the measurement accuracy for a specific component in a sample can be improved.

Next, a modification of the sample analysis device 6 will be described.

The first magnet unit 16 in the foregoing sample analysis device 6 is a semicircular shape. The first magnet unit 16 in the sample analysis device 6 according to the modification is a complete ring shape (hereinafter simply referred to as a “ring shape”).

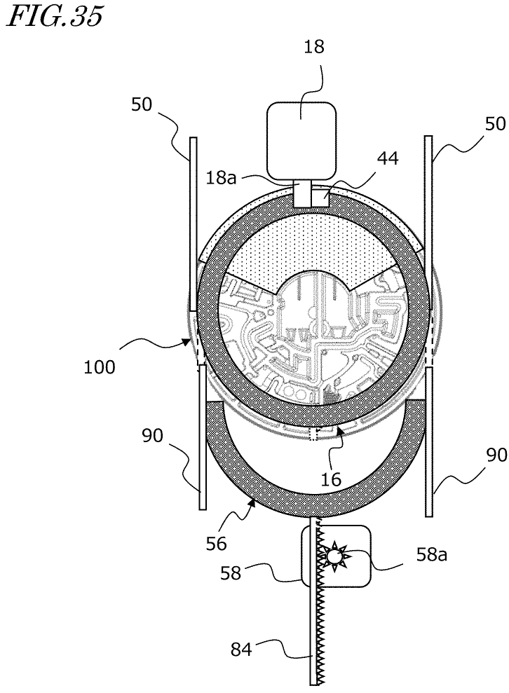

FIG. 35 shows an example relative positioning between a ring-shaped first magnet unit 16, a semicircular-shaped second magnet unit 56, and the sample analysis substrate 100. The ring of the first magnet unit 16 differs from the semicircular-shaped first magnet unit 16 (FIG. 24) only with respect to being changed to a circle; otherwise, it is identical to the example in FIG. 24. Therefore, even in this modification, the innermost periphery of the first magnet unit 16 passes through the substantial central position of the measurement chamber 116 regarding the radial direction. Moreover, the central position of the ring of the first magnet unit 16 regarding the radial direction matches the outermost position of the measurement chamber 116 regarding the radial direction. As a result, during rotation of the sample analysis substrate 100, the magnetic force (attractive force) of the first magnet unit 16 will always be applied to the position in the measurement chamber 116 where the magnetic particles gather the most. It will be appreciated that the magnitude of the attractive force per turn of the sample analysis substrate 100 is twice as that in the example of FIG. 24. This allows more magnetic particles to be adsorbed more promptly.

When the second magnet unit 56 moves to below the sample analysis substrate 100 for agitation, the first magnet unit 16 moves in parallel to the rotation axis of the sample analysis substrate 100, and goes away from the sample analysis substrate 100.

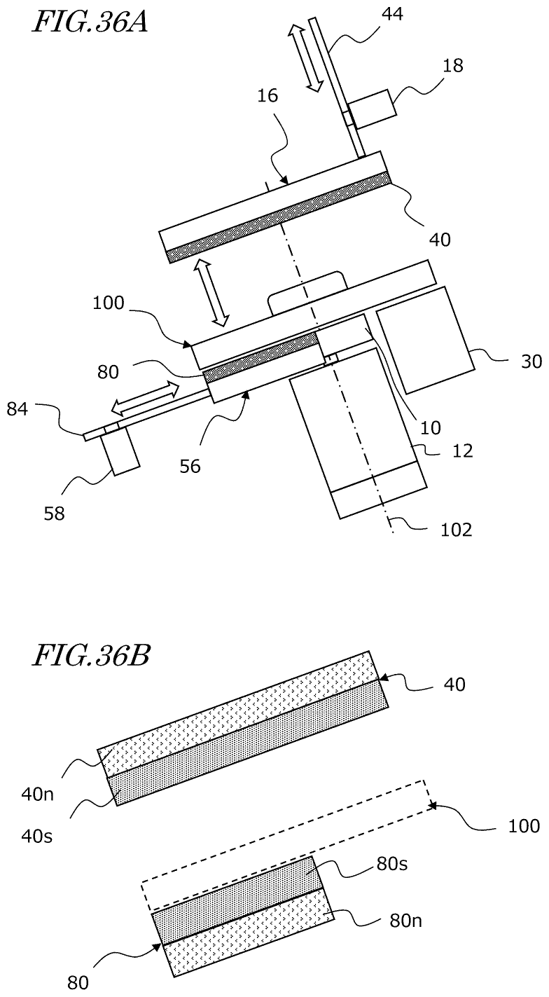

FIG. 36A is a side view of the sample analysis device 6 according to the modification. The positions of the first actuator 18 and the first rack 44 have been changed relative to the example of FIG. 31, in order to realize a movement of the first magnet unit 16 along a direction that is parallel to the rotation axis 102. The principle by which the first magnet unit 16 is moved is identical to that in the example of FIG. 31, and therefore the description thereof is omitted.

By adopting the ring-shaped first magnet unit 16, a portion of the first magnet unit 16 (a lower semicircular portion in FIG. 35) and the second magnet unit 56 are opposed to each other with the sample analysis substrate 100 interposed therebetween. Irrespective of the polarity of the magnet 40 of the first magnet unit 16 and the polarity of the magnet 80 of the second magnet unit 56, the magnetic particles 142 will be attracted to the first magnet unit 16 and to the second magnet unit 56. Therefore, the polarity of the magnet 40 of the first magnet unit 16 and the polarity of the magnet 80 of the second magnet unit 56 may be arbitrarily selected. However, the inventors have found that it is more preferable for the magnet 40 and the magnet 80 to have an identical polarity at the side where they face each other. This will be described below.

FIG. 36B is a schematic diagram for describing a relationship between the polarities of the magnet 40 and the magnet 80 in FIG. 36A. The position of the sample analysis substrate 100 is shown for referencing purposes.

In the present embodiment, as an example, the S-pole 40s of the magnet 40 is disposed so as to face toward the sample analysis substrate 100. The N-pole 40n of the magnet 40 is located on the opposite side to the S-pole 40s. On the other hand, the S-pole 80s of the magnet 80 is disposed so as to face toward the sample analysis substrate 100. The N-pole 80n of the magnet 80 is located on the opposite side to the S-pole 80s. In other words, the inventors have chosen to dispose the S-pole 40s of the magnet 40 and the S-pole 80s of the magnet 80 so that they face each other. The reason is that this allows the density of magnetic lines of force, i.e., the magnitude of the magnetic field, to be substantially zero.

This will be described more specifically below. The magnetic lines of force of the magnet 40 and the magnetic lines of force of the magnet 80 will never be connected. Therefore, when the S-pole 40s of the magnet 40 and the S-pole 80s of the magnet 80 face each other, even if their distance is not sufficiently large, i.e., even at a relatively close distance, the density of magnetic lines of force will be zero at a midpoint between the two magnets, thus zeroing the intensity of the magnetic field. Sometimes a demand may exist that no magnetic field be applied to a specific chamber or to any chamber. Establishing a zero magnetic field intensity at the midpoint between the two magnets 40 and 80 can meet such a demand. Because the distance between the two magnets 40 and 80 can be made relatively short, the size of the sample analysis device 6 can be kept compact. From the standpoint of substantially zeroing the magnetic field intensity between the two magnets 40 and 80, the N-pole 40n of the magnet 40 and the N-pole 80n of the magnet 80 may alternatively be made to face each other.

When the S-pole of the magnet 40 and the N-pole of the magnet 80 face each other, or when the N-pole of the magnet 40 and the S-pole of the magnet 80 face each other, the magnetic lines of force will pass through the midpoint between the two magnets. While securing a sufficiently long distance between the two magnets will allow the magnetic field intensity to become substantially zero, doing so will result in an increased size of the sample analysis device 6. Therefore, it is preferable that the S-poles or the N-poles of the magnet 40 and the magnet 80 face each other with the sample analysis substrate 100 being interposed therebetween, as described above.

The second magnet unit 56 may also be moved in a direction parallel to the rotation axis 102.

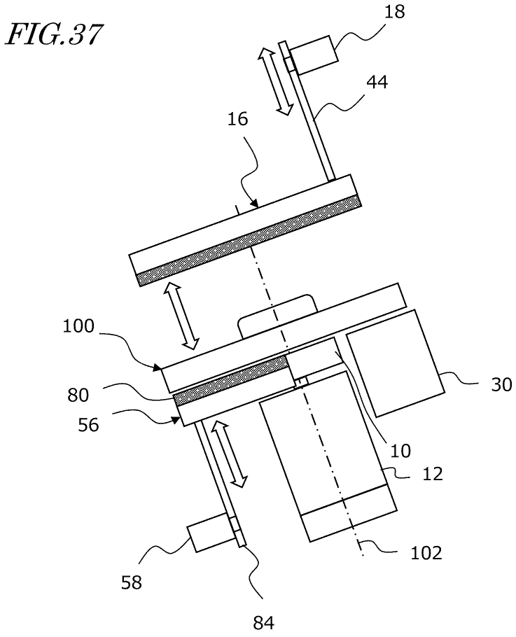

FIG. 37 is a side view of a sample analysis device 6 according to a further modification. The positions of the second actuator 58 and the second rack 84 have been changed relative to the example of FIG. 36A, in order to realize a movement of the second magnet unit 56 along a direction that is parallel to the rotation axis 102. The principle by which the second magnet unit 56 is moved is identical to that in the example of FIG. 35 and FIG. 36A, and therefore the description thereof is omitted.

The description of the above embodiment and its modifications has illustrated implementations where a pinion gear and a rack are utilized in order to drive each magnet unit. However, such implementations are only examples, and other mechanisms can also be used. For example, the magnet unit and the motor may be mechanically connected, and the position of the magnet unit may be changed with the rotational position of the motor, thus realizing retraction from the sample analysis substrate 100 and approach to the sample analysis substrate 100. In another example, the first magnet unit 16 and the second magnet unit 56 may be mechanically coupled so as to alternately retract from and approach the sample analysis substrate 100. A single actuator to replace the first actuator 18 and the second actuator 58 may be provided, and this actuator may be arranged so as to cause one magnet unit to retract from the sample analysis substrate 100 and cause the other magnet unit to approach the sample analysis substrate 100. The structure for moving one or more magnet units may as a whole be referred to as the “magnet moving mechanism”.

### INDUSTRIAL APPLICABILITY

A sample analysis device according to the present disclosure can be suitably used for at least one of: a B/F separation process; and agitation of magnetic particles and the sample, or luminescence measurement within a sample analysis substrate.

### REFERENCE SIGNS LIST

1: sample analysis device
2: housing
10: turntable
12: motor
14, 20: drive circuit
16: first magnet unit
18: first actuator
22: control circuit
30: photodetector
40, 40a to 40d: magnet
40e to 40h: group of magnets
42: case
56: second magnet unit
58: second actuator
100: sample analysis substrate
114: reaction chamber
116: measurement chamber
142: magnetic particles

1. A sample analysis device that rotates and stops a sample analysis substrate retaining a liquid sample to cause a binding reaction between an analyte in the liquid sample and a ligand immobilized to surfaces of magnetic particles,
the sample analysis substrate being capable of being mounted to or detached from the sample analysis device and including: a plate-shaped base substrate having a predetermined thickness; and a chamber within the base substrate, the chamber being a space in which to cause the binding reaction,
wherein the sample analysis device comprises:
a turntable to support the sample analysis substrate mounted thereon;
a motor to rotate the turntable;
a drive circuit to control rotation and stopping of the motor;
a first magnet unit to generate a force for attracting the magnetic particles;
a first actuator to move the first magnet unit to change relative positions of the first magnet unit and the sample analysis substrate; and
a control circuit to control operation of the motor, the drive circuit, and the first actuator, wherein the first magnet unit has a first shape that is a whole shape or a partial shape of a circle or a ring.

2. The sample analysis device of claim 1, wherein, during a B/F separation (Bound/Free Separation) for separating reacted substance from unreacted substance within the chamber, the first actuator moves the first magnet unit to a position where the magnetic particles in the chamber are attracted by the first magnet unit.

3. The sample analysis device of claim 2, wherein the first magnet unit comprises a single magnet having the first shape, or a plurality of magnets arranged along the first shape.

4. The sample analysis device of claim 1, wherein,
the sample analysis substrate is circular; and
the first shape of the first magnet unit is a whole or a part of the circle or the ring such that a sum of central angles thereof is not less than 90 degrees and not more than 360 degrees.

5. The sample analysis device of claim 1, wherein,
the sample analysis substrate is circular, and the first shape of the first magnet unit is a part of the circle or the ring; and
a length along a circumferential direction of the first magnet unit is longer than a length along a circumferential direction of the chamber.

6. The sample analysis device of claim 1, wherein,
the sample analysis substrate is circular; and
a radius size of the circle or the ring is determined in accordance with a distance from a center of rotation of the sample analysis substrate to the chamber.

7. The sample analysis device of claim 1, wherein,
the first shape of the first magnet unit is a whole or a part of the ring; and
the first actuator moves the first magnet unit during the B/F (Bound/Free) separation so that a central position regarding a radial direction of the ring matches a position in the chamber that is the farthest from the center of rotation of the sample analysis substrate.

8. The sample analysis device of claim 1, wherein the first actuator moves the first magnet unit along a direction that is parallel to a rotation axis of the sample analysis substrate.

9. The sample analysis device of claim 1, wherein the first actuator moves the first magnet unit along a direction that is perpendicular to a rotation axis of the sample analysis substrate.

10. The sample analysis device of claim 9, wherein the first actuator moves the first magnet unit to a position at which the first magnet unit and the sample analysis substrate do not overlap as viewed from a direction that is parallel to the rotation axis of the sample analysis substrate.

11. The sample analysis device of claim 1, wherein the first magnet unit is located on an opposite side of the sample analysis substrate from the turntable.

12. The sample analysis device of claim 1, wherein the first magnet unit is located on a same side of the sample analysis substrate as the turntable.

13. The sample analysis device of claim 2, further comprising:
a second magnet unit distinct from the first magnet unit; and
a second actuator to move the second magnet unit along a direction that is perpendicular to a rotation axis of the sample analysis substrate to change relative positions of the second magnet unit and the sample analysis substrate.

14. The sample analysis device of claim 13, wherein the second magnet unit comprises a single magnet having a second shape that is a whole shape or a partial shape of a circle or a ring, or a plurality of magnets arranged along the second shape.

15. The sample analysis device of claim 1, wherein the first actuator is a stepping motor or a linear motor.

16. The sample analysis device of claim 13, wherein the first actuator and the second actuator are a stepping motor(s) or a linear motor(s).

17. The sample analysis device of claim 13, wherein the first magnet unit is located on an opposite side of the sample analysis substrate from the turntable; and
the second magnet unit is located on a same side of the sample analysis substrate as the turntable.

18. The sample analysis device of claim 1, further comprising:
a second magnet unit to generate an attractive force for attracting the magnetic particles; and
a second actuator to move the second magnet unit to change relative positions of the second magnet unit and the sample analysis substrate, wherein,
the first magnet unit is disposed at a first face that is perpendicular to the rotation axis of the sample analysis substrate;
the second magnet unit is disposed at a second face that is perpendicular to the rotation axis of the sample analysis substrate, the second face being opposite to the first face;
the control circuit controls operation of the second actuator; and
during agitation of the liquid sample in the chamber, the first actuator and the second actuator alternately move the first magnet unit and the second magnet unit to a position where the magnetic particles in the chamber are attracted by the first magnet unit and the second magnet unit.

19. The sample analysis device of claim 18, wherein,
the first face is a face that is opposite to the turntable with respect to the sample analysis substrate; and
the second magnet unit has a second shape that is a partial shape of a circle or a whole shape or a partial shape of a ring.

20. The sample analysis device of claim 19, wherein,
the first magnet unit comprises a single magnet having the first shape or a plurality of magnets arranged along the first shape; and
the second magnet unit comprises a single magnet having the second shape or a plurality of magnets arranged along the second shape.

21. The sample analysis device of claim 19, wherein,
in a case where movement of the magnetic particles requires T seconds when the sample analysis substrate rotates at a predetermined number of revolutions and the magnetic particles are attracted at the number of revolutions;
in a period of 2T seconds, the first actuator causes the first magnet unit to approach the sample analysis substrate and move away from the sample analysis substrate, and,
in the period of 2T seconds, the second actuator causes the second magnet unit to move away from the sample analysis substrate and approach the sample analysis substrate.

22. The sample analysis device of claim 19, wherein,
the first shape and the second shape are a whole or a part of a ring;
the first actuator and the second actuator cause the first magnet unit and the second magnet unit, respectively, to approach the sample analysis substrate so that a central position regarding a radial direction of the ring matches a position in the chamber that is the farthest from a center of rotation of the sample analysis substrate.

23. The sample analysis device of claim 18, wherein the first actuator and the second actuator cause the first magnet unit and the second magnet unit, respectively, to move along a direction that is parallel to the rotation axis of the sample analysis substrate.

24. The sample analysis device of claim 19, wherein,
the second shape is a partial shape of the ring; and
the first actuator and the second actuator cause the first magnet unit and the second magnet unit, respectively, to move along a direction that is perpendicular to the rotation axis of the sample analysis substrate.

25. The sample analysis device of claim 24, wherein,
the first actuator causes the first magnet unit to move away to a position at which the first magnet unit and the sample analysis substrate do not overlap as viewed from a direction that is parallel to the rotation axis of the sample analysis substrate; and
the second actuator causes the second magnet unit to move away to a position at which the second magnet unit and the sample analysis substrate do not overlap as viewed from the direction that is parallel to the rotation axis of the sample analysis substrate.

26. The sample analysis device of claim 18, wherein,
the first magnet unit and the second magnet unit face each other with the sample analysis substrate interposed therebetween; and
N-poles or S-poles of the first magnet unit and the second magnet unit face each other.

27. The sample analysis device of claim 18, further comprising a photosensor disposed by the second face, wherein,
during a luminescence reaction to be effected by allowing a predetermined luminescent substrate to act on a composite of the analyte and the ligand being bound together after completion of the binding reaction;
the second actuator moves the second magnet unit to a position where the magnetic particles in the chamber are attracted by the second magnet unit; and
the photosensor detects light generated from the luminescence reaction.

28. The sample analysis device of claim 27, wherein the photosensor is a photomultiplier tube.

29. The sample analysis device of claim 18, wherein the first actuator and the second actuator are a stepping motor(s) or a linear motor(s).
